(19) United States
(12) Patent Application Publication
(10) Pub. No.: US 2025/0110119 A1
(43) Pub. Date: Apr. 3, 2025

(54) METHODS FOR QUANTITATING VIRAL CAPSIDS

(71) Applicant: Sartorius BioAnalytical Instruments, Inc., Bohemia, NY (US)

(72) Inventors: Yuanyuan Zhang, Newark, CA (US); Ling Zhang, Santa Clara, CA (US)

(73) Assignee: Sartorius BioAnalytical Instruments, Inc., Bohemia, NY (US)

(21) Appl. No.: 18/865,369

(22) PCT Filed: Feb. 28, 2024
(86) PCT No.: PCT/US2024/017641

§ 371 Date: Nov. 13, 2024

Related U.S. Application Data
(60) Provisional application No. 63/449,765, filed on Mar. 3, 2023.
(60) Provisional application No. 63/522,580, filed on Jun. 22, 2023.

(51) Int. Cl. G01N 33/53; C12Q 1/28; C12Q 1/30; C12Q 1/40; C12Q 1/42; C12Q 1/46; G01N 1/44; G01N 21/45

(52) U.S. Cl. CPC G01N 33/5308; C12Q 1/28; C12Q 1/30; C12Q 1/40; C12Q 1/42; C12Q 1/46; G01N 1/44; G01N 21/45; G01N 2333/015; G01N 2333/908; G01N 2333/916; G01N 2333/938

(57) ABSTRACT

Methods for quantitating viral capsids and/or polynucleotides contained therein using a charged biopolymer-loaded biosensor are provided. The signals indicative of binding of molecules (such as polynucleotides or viral capsids) to the biosensor can be measured by bio-layer interferometry (BLI). The amount of viral capsids can be calculated based on signals indicative of binding of the viral capsids to the biosensor. The empty/full viral capsid ratio in a viral sample can be calculated based on the amount of the viral polynucleotides and/or the binding kinetics of the biosensor to the viral capsids or the polynucleotides.

### CROSS-REFERENCE TO RELATED APPLICATION

This application is a national stage filing under 35 U.S.C. § 371 of International Patent Application No. PCT/US2024/017641, filed Feb. 28, 2024, which claims priority to both U.S. Provisional Application No. 63/449,765, filed Mar. 3, 2023, and U.S. Provisional Application No. 63/552,580, filed Jun. 22, 2023, the entire contents of which are hereby incorporated by reference.

### BACKGROUND

The present disclosure relates to methods for quantitating viral capsids and for determining empty/full ratios on viral capsids using an optical sensor.

Viral vectors, such as adeno-associated virus (AAV) vectors, for gene therapy may provide a durable treatment response for a number of diseases with unmet needs. Viral vectors can be used for delivery of polynucleotides (such as DNA) encoding genes intended to treat a variety of diseases. The natural byproducts of recombinant virus synthesis are capsids that have not been packaged with polynucleotides of interest, also referred to as “empty” capsids, and capsids that have been packaged with polynucleotides of interest, also referred to as “full” capsids. Removal of empty capsids can increase transgene expression in subjects. Accordingly, it is important to have effective methods to differentiate full capsids from empty capsids. Despite the purification steps that have been integrated into the recombinant virus (such as recombinant AAV) production chain, empty capsids can still account for a significant portion of a batch.

Several methods have been utilized in attempt to determine the capsid content ratio (full vs empty capsids), including anion-exchange chromatography (AEX), optical density (OD), transmission electron microscopy (TEM), charge detection mass spectrometry (CDMS), size-exclusion chromatography multiangle light scattering (SEC-MALS), enzyme-linked immunosorbent assay (ELISA) in tandem with quantitative polymerase chain reaction (qPCR), analytical ultracentrifugation (AUC), dynamic/static light scattering (DLS/SLS), and microfluidic technology. However, these techniques are time-consuming, laborious, error-prone, inaccurate, expensive, and limited by their requirement of significant sample preparation or processing times. For example, sample analysis times for these techniques can range between 15 min (OD) and 6 h (AUC and TEM) per sample, and require extensive resources, which significantly limit the number of samples that can be processed in a day and renders most approaches as low throughput.

### SUMMARY

In view of the foregoing, there is a need for methods that efficiently, accurately, and rapidly measure the amount of viral capsids, polynucleotides contained therein, and/or empty/full capsid ratios. This disclosure is directed generally to systems and methods to address these shortcomings of the art and provide other additional or alternative advantages. The disclosure herein provides embodiments of systems and methods for quantitating viral capsids and/or polynucleotides contained therein using a biosensor. The signals indicative of binding of molecules (such as polynucleotides or viral capsids) to the biosensor can be measured by an interferometer based on bio-layer interferometry (BLI). The empty/full viral capsid ratio in a viral sample can be calculated based on the binding signal intensity or binding kinetics of the biosensor to the viral capsids or the polynucleotides.

In certain aspects of the present disclosure, a method for quantitating polynucleotides contained within one or more viral capsids in a sample is provided. The method includes the steps of: subjecting the sample containing viral capsids, which contain full viral capsids (containing polynucleotides) and/or empty viral capsids (not containing polynucleotides), to a separation process to separate the polynucleotides from the viral capsids; contacting the polynucleotides with a biosensor to bind the polynucleotides to a positively charged biopolymer of the biosensor; and contacting signal development elements with the polynucleotides bound to the biosensor to generate signals indicative of an amount of the polynucleotides bound to the biosensor. The biosensor contains a core component, a negatively charged biopolymer bound to an external surface of the core component, and the positively charged biopolymer bound to an external surface of the negatively charged biopolymer such that the viral capsids and polynucleotides can bind to the positively charged biopolymer.

In embodiments, the step of separating the polynucleotides from the viral capsids can include applying heat to the viral capsids and releasing the polynucleotides from the viral capsids. The polynucleotides may comprise single stranded DNA (ssDNA). The viral capsids may comprise capsids of adeno-associated virus (AAV). The AAV may include AAV serotype 2 (AAV2) or AAV serotype 8 (AAV8).

In some embodiments, the core component on the biosensor comprises or epoxypropylsilane (EPS) or aminopropylsilane (APS). In some embodiments, the negatively charged biopolymer of the biosensor comprises of DNA, dextran, a carboxylic acid (COOH)-functionalized compound, and/or other polysaccharide compounds. The positively charged biopolymer may comprise of polyethylenimine (PEI), chitosan, poly-L-lysine, polyallylamine, polyaziridine, and/or other amine-functionalized compounds.

In certain embodiments, the signal development elements comprise a dye, an antibody, or an antibody linked to a first enzyme. The dye may be a cyanine dye, ethidium bromide, propidium iodide, crystal violet, a dUTP-conjugated probe, DAPI (4′,6-diamidino-2-phenylindole), 7-aminoactinomycin D (7-AAD), a Hoechst dye (such as Hoechst 33258, Hoechst 33342, Hoechst 34580), a CYBR® dye (such as CYBR® Gold, CYBR® Green, CYBR® Safe), or EVAGREEN®. The dye may be a cyanine dye. The antibody may be a nucleic acid antibody, a single stranded DNA (ssDNA) antibody, and/or a single stranded/double stranded DNA (ss/dsDNA) specific antibody. The enzyme may be horseradish peroxidase (HRP), alkaline phosphatase (AP), β-galactosidase, acetylcholinesterase, or catalase.

In embodiments, the signals are generated by contacting the one or more signal development elements comprising the antibody linked to the first enzyme with a substrate. In other embodiments, the signals are generated by contacting the one or more signal development elements comprising the antibody with a secondary antibody linked to a second enzyme, and then contacting the second enzyme with a substrate.

In particular embodiments, the method provided herein comprises quantitating polynucleotides contained within one or more viral capsids in a subject sample (with an unknown viral titer and/or empty/full viral capsidratio) or a reference sample (with a known viral titer and/or empty/full viral capsid ratio). The method may further comprise determining a ratio of empty and full viral capsids based at least partially on the amount of polynucleotides in the reference sample bound to the biosensor and an amount of polynucleotides in the subject sample bound to the biosensor.

In certain embodiments, different full/empty viral capsid ratios in samples results in different binding kinetics of the biosensor to the polynucleotides that are present or absent in the samples. The method may comprise determining a ratio of empty and full viral capsids based at least partially on the binding kinetics of the biosensor to the polynucleotides, for example in the reference sample and/or the subject sample.

In embodiments, the signals are indicative of wavelength/spectral/nm shift measured using bio-layer interferometry (BLI). In embodiments, the signals generated by a positive shift indicates binding of the one or more polynucleotides to the biosensor.

In some embodiments, the method further includes the step of contacting a sample containing viral capsids with an affinity biosensor to bind viral capsids, for example to isolate or purify viral capsids from a crude sample, before the step of separating the polynucleotides from viral capsids. An affinity biosensor used herein can be an antibody-based affinity biosensor.

In certain aspects of the present disclosure, a method for quantitating viral capsids (such as AAV viral capsids) in a sample is provided. The method includes the steps of: contacting the viral capsids, which contain full viral capsids (containing polynucleotides) and/or empty viral capsids (not containing polynucleotides), with a biosensor to bind viral capsids to a positively charged biopolymer of the biosensor and generate a first set of signals indicative of binding of the viral capsids to the biosensor; and quantitating the first set of signals generated by binding of the viral capsids to the biosensor. The biosensor contains a core component, a negatively charged biopolymer bound to an external surface of the core component, and the positively charged biopolymer bound to an external surface of the negatively charged biopolymer such that the viral capsids can bind to the positively charged biopolymer.

In embodiments, the first set of signals are indicative of an amount of the viral capsids bound to the biosensor and/or a full/empty viral capsid ratio based at least partially on binding kinetics of the biosensor to the viral capsids. In certain embodiments, the first set of signals are measured using BLI.

In certain embodiments, different full/empty viral capsid ratios in samples result in different binding kinetics of the biosensor towards the viral capsids contained in the samples. The method may further comprise determining a ratio of empty and full viral capsids based at least partially on the binding kinetics of the biosensor towards the viral capsids.

In embodiments, the first set of signals are indicative of wavelength/spectral/nm shift measured using bio-layer interferometry (BLI). In embodiments, the first set of signals generated by a positive shift indicates binding of the one or more polynucleotides to the biosensor.

In further embodiments, the method can further include the steps of subjecting the one or more viral capsids bound to the biosensor to a separation process to separate the polynucleotides from the one or more viral capsids, contacting the polynucleotides with the biosensor to bind the polynucleotides to the positively charged biopolymer of the biosensor, and contacting the polynucleotides bound to the biosensor with signal development elements to generate a second set of signals indicative of an amount of the polynucleotides bound to the biosensor.

In particular aspects of the present disclosure, a method for quantitating polynucleotides contained within viral capsids in a sample is provided. The viral capsids may be those of adeno-associated virus (AAV), for example adeno-associated virus type 2 (AAV2) and adeno-associated virus type 8 (AAV8). The method includes the steps of: contacting the sample, which contain full viral capsids (containing polynucleotides) and/or empty viral capsids (not containing polynucleotides), with a first biosensor, which is an affinity biosensor, to bind viral capsids; subjecting the (full and/or empty) viral capsids bound to the affinity biosensor to a separation process to separate the polynucleotides from the viral capsids; contacting the polynucleotides with a second biosensor to bind the polynucleotides to the biosensor; contacting the polynucleotides bound to the second biosensor with signal development elements (such as a dye, such as a cyanine dye; or an antibody, such as a nucleic acid antibody, a ssDNA antibody, or a ss/dsDNA specific antibody) to generate signals indicative of an amount of the polynucleotides bound to the biosensor; and determining a ratio of empty and full viral capsids based at least partially on the amount of polynucleotides (in a subject sample and/or a reference sample) bound to the second biosensor, and/or binding kinetics of the second biosensor to the polynucleotides (in a subject sample and/or a reference sample). The first biosensor may be an AAV antibody-based affinity biosensor. The second biosensor may be a biopolymer-based biosensor, and may contain a core component, a negatively charged biopolymer bound to an external surface of the core component, and the positively charged biopolymer bound to an external surface of the negatively charged biopolymer such that the viral capsids and/or polynucleotides can bind to the positively charged biopolymer.

In particular embodiments, the biosensor comprises an optical fiber having a proximal end portion and a distal end portion. The proximal end portion may be configured to receive light from a light source and configured to deliver reflected light to a detector. The distal end portion may be configured to have analytes bind thereto such that light reflected from the distal end portion is phase shifted based on a thickness of analytes bound to the distal end portion. The biosensor may further comprise an optical resonator at a distal end portion of the optical fiber. The optical resonator includes a first reflective surface and a second reflective surface. The first reflective surface may be configured to reflect light with a first phase and the second reflective surface may be configured to reflect light with a second phase which is phase shifted based on a thickness of analytes bound to the optical resonator.

In certain embodiments of the methods provided herein, the signals such as those generated by binding of the signal development elements to the polynucleotides and/or those generated by binding of the viral capsids to the biosensor are measured by an interferometer. The interferometer may comprise the biosensor. The interferometer may further include: a first optical waveguide configured to receive light from a light source, a second optical waveguide configured to deliver reflected light to a detector, and an optical coupler, attached to the biosensor, that spatially separates a distal portion of the first optical waveguide from a distal portion of the second optical waveguide. The interferometer may further comprise a light source that is in optical communication with the first optical waveguide and configured to provide light to the first optical waveguide. The interferometer may further comprise a detector configured to receive light from the second optical waveguide. The first optical waveguide and the second optical waveguide may be disposed in a fiber optic bundle.

### BRIEF DESCRIPTION OF THE DRAWINGS

Various aspects of the present disclosure are described hereinbelow with reference to the drawings, which are incorporated in and constitute a part of this specification.

FIGS. 1A and 1B are diagrammatic representations of a method of separating viral capsids and polynucleotides, such as single strand DNA (ssDNA), contained therein. FIG. 1A depicts purifying viral capsids from a crude sample by contacting the viral capsids with an affinity biosensor, allowing the viral capsids to bind to the biosensor, followed by applying heat stress to the viral capsids, and releasing the polynucleotides (such as ssDNA) from the viral capsids having been bound to the biosensor. FIG. 1B depicts separating viral capsids and polynucleotides (such as ssDNA) in a purified sample by applying heat stress.

FIGS. 2A and 2B schematically depict an example method of capturing and quantitating viral polynucleotides (such as ssDNA) using bio-layer interferometry (BLI), wherein a biosensor is loaded with charged biopolymers. FIG. 2A includes a negatively charged biopolymer loading phase and a positively charged biopolymer loading phase. FIG. 2B includes a positively charged biopolymer loading phase onto a biosensor preloaded with negatively charged biopolymer.

FIGS. 3A-3H depict interferometry signal change (nm shift) that is indicative of the binding rate of samples each with a certain empty/full capsid ratio of a population of adeno-associated virus type 8 (AAV8). FIGS. 3A and 3B depict the signal change over an example process comprising the preloaded negatively charged biopolymer equilibrium phase, the positively charged biopolymer loading phase, the polynucleotide capture phase, the signal amplification phase, and washes in between. In FIG. 3A, the sample contained 1×10¹¹ vg/ml of AAV8, and a dye is used for signal amplification. In FIG. 3B, the sample contained 2.5×10¹¹ vg/ml of AAV8, and an antibody is used for signal amplification. FIGS. 3C and 3D depict the polynucleotide capture phase of FIGS. 3A and 3B, respectively. FIGS. 3E and 3F depict dye- and antibody amplification-based signal amplification phases, respectively, according to the embodiments of the present disclosure. FIGS. 3G and 3H depict the initial slope and end-point signal changes (indicative of binding rates) using a dye and an antibody, respectively, for amplification, for the AAV8 samples with different empty/full capsid ratio studied in FIGS. 3A-3F.

FIG. 4 schematically depicts a method for empty and full capsid (e.g., AAV capsid) differentiation using BLI. A secondary antibody, which is linked to (such as conjugated with) an enzyme (e.g., HRP), binds to a primary antibody (or dye) that is bound to ssDNA released from an AAV capsid. HRP catalyzes the conversion of a substrate for the detection of ssDNA or other polynucleotides.

FIG. 5 depicts a graph of an interferometry signal shift (nm) that is indicative of the amount of samples each with a certain empty/full capsid ratio of a population of AAV8 using the method illustrated in FIG. 4. The signal change is depicted over an example process comprising the preloaded negatively charged biopolymer equilibrium phase, the positively charged biopolymer loading phase, the polynucleotide capture phase, the detection antibody (or dye) binding phase, the HRP-linked secondary antibody binding phase, signal amplification phase from HRP catalyzing the conversion of the substrate, and washes in between.

FIG. 6 schematically depicts a method for empty and full capsid (e.g., AAV capsid) differentiation using BLI. A detection antibody, which is linked to (such as conjugated with) an enzyme (e.g., HRP), binds directly to ssDNA. No secondary antibody is needed.

FIG. 7 depicts a graph of an interferometry signal shift (nm) that is indicative of the amount of samples each with a certain empty/full capsid ratio of a population of AAV8 using the method illustrated in FIG. 6. The signal change is depicted over an example process comprising the preloaded negatively charged biopolymer equilibrium phase, the positively charged biopolymer loading phase, the polynucleotide capture phase, the HRP-linked capture antibody binding phase, signal amplification phase from HRP catalyzing the conversion of the substrate, and washes in between.

FIG. 8 is a graphical representation of the direct viral titer measurement based on direct binding of AAV8 full capsids to a BLI biosensor loaded with charged biopolymers. The BLI biosensor loaded with charged biopolymers was contacted with samples having different titer of AAV8 full capsids, and the signal change (nm shift) was measured as an indicator of the amount of AAV8 full capsids bound to the BLI biosensor over an example process comprising the preloading phase, the negatively charged biopolymer loading phase, the positively charged biopolymer loading phase, and the capsid capture phase.

FIG. 9 is a graphical representation of the direct empty/full ratio measurement based on direct binding of AAV2 full capsids to a BLI biosensor loaded with charged biopolymers. The BLI biosensor loaded with charged biopolymers was contacted with AAV2 samples having the same titer, but different empty/full capsid ratios, and the signal change (nm shift) was measured as an indicator of the amount of AAV2 capsids bound to the BLI biosensor over an example process comprising the preloaded negatively charged biopolymer equilibrium phase, the positively charged biopolymer loading phase, and the capsid capture phase.

FIG. 10 is a flowchart of a method of quantitating a ratio of polynucleotides contained within the viral capsids in a sample according to embodiments of the present disclosure.

FIG. 11 is a flowchart of a method of quantitating an amount of viral capsids contained within a sample according to embodiments of the present disclosure.

### DETAILED DESCRIPTION

The present disclosure will now be described more fully hereinafter with reference to example embodiments thereof and with reference to the drawings. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements.

A “vector,” as used herein, refers to a recombinant plasmid or virus that comprises a nucleic acid to be delivered into a host cell, either in vitro or in vivo. A “polynucleotide” as used herein refers to a biopolymer comprising a plurality of nucleotide monomers, covalently bonded in a chain, including DNA (deoxyribonucleic acid) and RNA (ribonucleic acid). Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

Viral titers can be expressed in a number of ways, from which one skilled in the art can select a way to express viral titers suitable in the context. For example, the term “capsid particles (cp)” as used in reference to a viral titer refers to the number of viral capsids, regardless of infectivity or functionality. The terms “viral genomes (vg),” “genome particles,” “genome equivalents,” or “genome copies” as used in reference to a viral titer, refer to the number of virions containing the recombinant viral DNA genome or RNA genome, regardless of infectivity or functionality. The number of capsid particles or genome particles in a particular vector preparation can be measured by standard methods such as using a fluorescent dye or electron microscopy. The terms “infection unit (iu),” “infectious particle,” or “replication unit,” as used in reference to a viral titer, refer to the number of infectious and replication-competent recombinant viral vector particles as measured by the infectious center assay, also known as replication center assay. The term “transducing unit (tu),” as used in reference to a viral titer, refers to the number of infectious recombinant viral vector particles that result in the production of a functional transgene product as measured in functional assays.

The terms “isolate” or “isolating” a molecule (such as polynucleotide or viral capsid) as used herein refers to identifying and separating and/or recovering the molecule from a component of its natural environment. Thus, for example, isolated (or separated or purified) viral particles may be prepared using a purification technique to enrich them from a source mixture, such as a culture lysate or production culture supernatant. Similarly, isolated (or separated or purified) viral polynucleotides may be prepared using a purification technique to separate them from a source mixture that contains viral capsids.

The term “load” or “loaded” in the context of loading a biopolymer to a structure to assemble a biosensor refers to the process of bringing the equilibrated sample (such as biopolymer) into contact with the equilibrated solid phase (such as to assemble a biosensor). Loading can be done for example with chromatography devices by causing the sample to pass through the device by means of an external force, such as by gravity or by pumping, or by dipping into a well plate that contains the sample.

As used herein, the term “BLI biosensor” refers to a sensing and/or analytical device that detects the presence, physical characteristics, or amount of substances using a biological molecule (such as an enzyme or an antibody) or a living organism suitable for BLI detection. The biosensor has a distal end and a proximal end. The proximal end has a surface coated with a thin layer of analyte-binding molecules.

As used herein, the term “proximal” refers to the portion of the device or component thereof that is closer to the user or machine using the device and the term “distal” refers to the portion of the device or component thereof that is farther from the user or machine using the device.

An “analyte-binding” molecule refers to any molecule capable of participating in a specific binding reaction with an analyte molecule. Examples include, but are not limited to, antibody-antigen binding reactions and nucleic acid hybridization reactions.

An “antibody” refers to an immunoglobulin molecule having two heavy chains and two light chains prepared by any method known in the art or later developed and includes polyclonal antibodies such as those produced by inoculating a mammal such as a goat, mouse, rabbit, etc. with an immunogen, as well as monoclonal antibodies produced using the well-known Kohler Milstein hybridoma fusion technique. The term includes antibodies produced using genetic engineering methods such as those employing, e.g., SCID mice reconstituted with human immunoglobulin genes, as well as antibodies that have been humanized using art-known resurfacing techniques. An antibody also refers to an antibody fragment. An “antibody fragment” refers to a fragment of an antibody molecule produced by chemical cleavage or genetic engineering techniques, as well as to single chain variable fragments (SCFvs) such as those produced using combinatorial genetic libraries and phage display technologies. Antibody fragments used in accordance with the present disclosure usually retain the ability to bind their cognate antigen and so include variable sequences and antigen combining sites, which are within the scope of antibodies.

As used herein, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like. Reference to a value or parameter herein includes (and describes) embodiments that are directed to that value or parameter per se with in a range suitable in the context, for example ±10%. For example, description referring to “X” includes description of “X” and extends to a suitable range.

A “binding rate” as used herein refers to a computational output indicative of the quality and nature of biomolecules or complexes thereof binding to a biosensor.

A method of the present disclosure for quantitating polynucleotides contained within one or more viral capsids in a sample can include the steps of separating the polynucleotides from the one or more viral capsids, contacting the polynucleotides with a biosensor, contacting the polynucleotides bound to the biosensor with signal development elements to bind the signal development elements to the polynucleotides and generate signals, and measuring generated signals, which are indicative of an amount of polynucleotides bound to the biosensor.

The biosensor comprises a core component, a negatively charged biopolymer bound to an external surface of the core component, a positively charged biopolymer bound to an external surface of the negatively charged biopolymer. Upon contacting with the biosensor, the polynucleotides bind to the positively charged biopolymer of the biosensor. The polynucleotides provided herein can comprise single stranded DNA.

Non-limiting examples of viral capsids that can be evaluated using the methods and systems disclosed herein include viral capsids from retroviruses, adenoviruses, adeno-associated viruses, lentiviruses, and herpes simplex viruses. In some embodiments, the one or more viral capsids comprise capsids of adeno-associated virus (AAV) including recombinant AAV. A “recombinant” virus or “recombinant” viral vector as used herein refers to a virus or polynucleotide vector comprising one or more heterologous sequences (in other words, nucleic acid sequence not of viral origin). A “viral capsid” as used herein includes either a protein shell of viral protein, or a lipid bilayer coat/envelop, that encloses viral nucleic acid or recombinant viral vector. A “full” viral capsid as used herein refers to a viral capsid that contains polynucleotides. An “empty” viral capsid as used herein refers to a viral capsid that does not contain polynucleotides, or that is essentially free of polynucleotides. “Essentially free” as used herein in reference to a particular component (such as polynucleotides) means that the component (such as polynucleotides) present constitutes less than about 3.0% by weight, such as less than about 2.0% by weight, less than about 1.0% by weight, less than about 0.5% by weight or less than about 0.1% by weight of the composition (such as a viral capsid).

An “empty/full” viral capsid ratio refers to the ratio of empty viral capsids to full viral capsids in a sample, or in a population of viral capsids.

AAV is a single-stranded DNA (ssDNA) nonenveloped virus that belongs to the parvovirus family and measures 25 nm in diameter. There are at least eleven serotypes of AAV (AAV1, AAV2, AAV3, AAV4, AAV5, AAV6, AAV7, AAV8, AAV9, AAV10, AAV11), each with slightly different tropisms which include retina, lung, muscle, liver, and brain cells. The virus is only composed of protein and DNA and has three repeating capsid proteins, VP1, VP2, and VP3, at an expected ratio of 1:1:10, which may vary across serotypes and even within the particles of a given batch. It is capable of infecting both dividing and nondividing cells depending on the tropism of the given serotype. In some embodiments, the AAV provided herein is AAV serotype 2 (AAV2), AAV serotype 8 (AAV8), or AAV serotype 9 (AAV9).

The simplicity of the AAV genome makes recombinant AAV (rAAV) a great candidate for gene delivery. While AAV generally requires a helper virus, such as an adenovirus or herpes simplex virus, to successfully replicate in human cells, a key change in the development of its design was the use of plasmids for rAAV production. A common method is the use of the triple transfection of HEK293 cells, where one plasmid includes the gene of interest, another the helper genes, and a third the packaging genes. The integration of the helper virus functional genes allows for the helper virus-independent manufacturing of recombinant AAV vectors. AAV vector can also be produced in Sf9 cells through infection with two recombinant baculoviruses, a first one carrying the AAV rep/cap genes, and a second carrying the gene of interest flanked by two AAV ITRs. Additionally, while its restricted packaging limit of 4.7 kb previously limited its scope, the potential and integrity of oversized rAAV vectors has been explored. Capsid-modified, genome-modified, and both capsid- and genome-modified vectors are further being developed to increase their efficiencies at reduced doses.

In some embodiments, the methods provided herein can comprise contacting a sample containing polynucleotides (such as viral polynucleotides released from viral capsids) with a biosensor such that the polynucleotides are bound to the biosensor. The biosensor used according to the methods provided herein can comprise a core component, a negatively charged biopolymer bound to an external surface of the core component, and a positively charged biopolymer bound to an external surface of the negatively charged biopolymer. In the methods provided herein, upon contacting with the biosensor, the polynucleotides (such as ssDNA separated from the viral capsids) bind to the positively charged biopolymer of the biosensor. Without wishing to be bound by theory, the multi-layer structure (comprising at least a core component, a negatively charged biopolymer layer, and a positively charged biopolymer layer) provides stability to the biosensor. Any suitable material can be used for the core component, the negatively charged biopolymer layer, and the positively charged biopolymer layer. For example, the core component can comprise a silicon substrate, activated with silane group, such as aminopropylsilane (APS) and epoxypropylsilane (EPS). The negatively charged biopolymer of the biosensor can comprise DNA, dextran, a carboxylic acid (COOH)-functionalized compound (such as beads), and/or other polysaccharide compounds. The positively charged biopolymer of the biosensor can comprise polyethylenimine (PEI), chitosan, poly-L-lysine, polyallylamine, polyaziridine, and/or other amine-functionalized compounds. In some embodiments, the components of the biosensor, such as the core component, the negatively charged biopolymer layer, and the positively charged biopolymer layer are non-toxic.

Solid surfaces can be activated and used to generate a biosensor provide herein. For example, layer-by-layer (LbL) assembly assembles a thin film by depositing (or loading) alternating layers of oppositely charged materials with wash steps in between. The LbL assembly technique can generate ultrathin films with nanoscale thickness, orientation control with molecular order and stability which involves the alternate adsorption of oppositely charged layers of polyions and biopolymers involving variety of organic and inorganic substrate materials including carbon nanotubes (CNTs), proteins, antigens, lysozyme, DNA, nanoparticles, metallophthalocyanines, and dendrimers. The LbL assembly technique is also a versatile method for fabrication of controlled layered structures from CNT/enzyme using simple, rapid and inexpensive procedures, and is useful for immobilization of biopolymers, biocatalysts, and biomaterials in well-defined layered structures under mild conditions rendering for biosensor and biofuel cell applications. In specific embodiments, the biosensor provided herein is generated using the LbL assembly technique.

Full viral capsids contain polynucleotides, such as therapeutic polynucleotides. Polynucleotides can be separated from viral capsids. In some embodiments, the step of separating comprises applying heat (heat stress) to the viral capsids. Heat stress releases the polynucleotides from the viral capsids. For example, the viral capsids, or a sample containing the viral capsids, can be heated to a temperature between 9° and 95° C. (such as 90, 91, 92, 93, 94, or 95° C.) for 2-5 minutes (such as 2, 3, 4, or 5 minutes) to release and separate polynucleotides from the viral capsids.

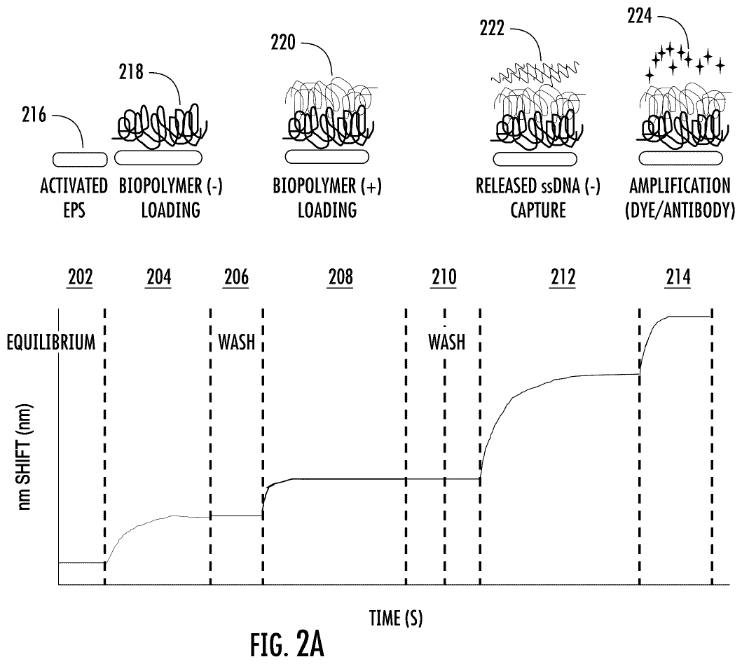

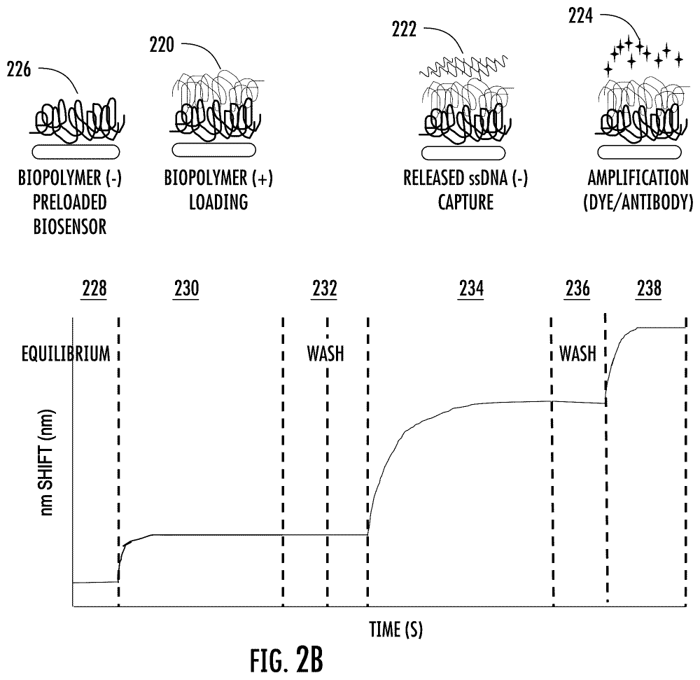

FIGS. 2A and 2B schematically depict embodiments of a method of capturing and quantitating viral polynucleotides (such as ssDNA) using a bio-layer interferometry (BLI) biosensor loaded with charged biopolymers. The shift in interferometry signal (“nm shift”) over time indicates binding of a material (such as biopolymer or ssDNA) to the BLI biosensor. The signals can be indicative of wavelength/spectral/nm shift measured using bio-layer interferometry (BLI). A positive shift can indicate binding of the one or more polynucleotides to the biosensor. The process can comprise different phases. In FIG. 2A, the example phases include an equilibrium 202, a negatively charged biopolymer loading phase 204, wash 206, a positively charged biopolymer loading phase 208, washes 210, a polynucleotide capture phase 212, and a signal amplification phase 214 conducted sequentially in this order. In FIG. 2B, the example phases include an equilibrium 228, a positively charged biopolymer loading phase 230, washes 232, a polynucleotide capture phase 234, a wash 236, and a signal amplification phase 238 conducted sequentially in this order.

The first phase 202 of FIG. 2A depicts an embodiment of an equilibrium in which the core component of the BLI biosensor 216 is EPS (epoxypropylsilane) activated. “EPS activated” as used herein refers to epoxy silane on the surface.

The second phase 204 of FIG. 2A depicts an embodiment of a negatively charged biopolymer loading phase. Negatively charged biopolymer 218 is bound to the EPS activated 216 of the core component. The third phase 206 of FIG. 2A depicts wash with an appropriate buffer. The fourth phase 208 of FIG. 2A depicts an embodiment of positively charged biopolymer loading phase. Positively charged biopolymer 220, such as PEI is bound to the negatively charged biopolymer 218. The fifth and sixth phases 210 of FIG. 2A depict wash with an appropriate buffer.

The seventh phase 212 of FIG. 2A depicts an embodiment of a polynucleotide capture phase. The sample containing polynucleotides 222 (such as ssDNA) released and separated from viral capsids is contacted with the BLI biosensor, and the polynucleotides 222 and/or viral capsids bind to the positively charged biopolymer 220 of the biosensor. Polynucleotides, such as ssDNA, and viral capsids are negatively charged, and therefore they bind to the positively charged biopolymer layer.

The eighth phase 214 of FIG. 2A depicts an embodiment of a signal amplification phase. The polynucleotides 222 bound to the biosensor are contacted with signal development elements 224, which bind to the polynucleotides. A “signal development element” as used herein refers to a molecule that binds to the polynucleotides and develops a signal with intensity that correlates with the amount of polynucleotides present (bound to the biosensor). Any signal development element can be used, including a dye that bind to polynucleotides (such as a DNA dye), an antibody (such as an DNA antibody), or an aptamer, or an antibody linked to a detectable label (such as an enzyme, luciferase, a fluorescent label), or an antibody for a sandwich assay in which a second antibody with a detectable label is bound to the first antibody. In sandwich assays, the first and second antibodies can be the same (particularly when polyclonal antibodies are used) or different (for example, an antibody and another binding entity that is not a monoclonal antibody, such as a polyclonal antibody or an aptamer). Non-limiting examples of enzymes that can be used as a signal development element include horseradish peroxidase (HRP), alkaline phosphatase (AP), β-galactosidase, acetylcholinesterase, and catalase. Non-limiting examples of substrates that can be used with both of the above methods include chromogenic and chemiluminescent substrates, such as: 3,3′-Diaminobenzidine (DAB), metal DAB, 3, 3′, 5, 5′-tetramethylbenzidine (TMB), chlorophenol, chloronaphthol, HISTOMARK® TRUEBLUE™ Peroxidase Substrate, HISTOMARK® BLACK peroxidase system, HISTOMARK® ORANGE, LUMIGLO® chemiluminescent substrate, LUMIGLO® reserve chemiluminescent substrate, SG/IMMPACT® SG, VIP/IMMPACT® VIP, and NOVARED®/IMMPACT NOVARED® as HRP substrates; and nitro blue tetrazolium (NBT)/5-bromo-4-chloro-3-indolyl-phosphate/nitro blue tetrazolium (BCIP), VECTOR® Red, VECTOR® Blue, and VECTOR® Black as AP substrates.

In some embodiments where an enzyme (such as HRP, AP, β-galactosidase, acetylcholinesterase, catalase) linked to an antibody is used to quantitate polynucleotides bound to the BLI biosensor, the signal amplification phase can include multiple steps for binding of antibodies and development of signals catalyzed by the enzyme. Such embodiments are described further below.

As compared to FIG. 2A, FIG. 2B does not have a negatively charged biopolymer loading phase. The first phase 228 of FIG. 2B depicts an embodiment of an equilibrium of a biosensor 226 in which a negatively charged biopolymer is preimmobilized onto a core component. The second phase 230 of FIG. 2B depicts an embodiment of positively charged biopolymer loading phase. Positively charged biopolymer 220, such as PEI is bound to the negatively charged biopolymer 218. The third and fourth phases 232 of FIG. 2B depict wash with an appropriate buffer.

The fifth phase 234 of FIG. 2B depicts an embodiment of a polynucleotide capture phase. The sample containing the polynucleotides 222 (such as ssDNA) released and separated from viral capsids is contacted with the BLI biosensor, and the polynucleotides 222 and/or viral capsids bind to the positively charged biopolymer 220 of the biosensor. The sixth phase 236 of FIG. 2B depict wash with an appropriate buffer.

The seventh phase 238 of FIG. 2B depicts an embodiment of a signal amplification phase. The polynucleotides 222 bound to the biosensor are contacted with signal development elements 224, which bind to the polynucleotides.

In some embodiments, the signal development elements comprise a molecule, such as a dye that specifically binds to the ssDNA. In some embodiments, the dye is selected from the group consisting of a cyanine dye, ethidium bromide, propidium iodide, crystal violet, a dUTP-conjugated probe, DAPI (4′,6-diamidino-2-phenylindole), 7-aminoactinomycin D (7-AAD), a Hoechst dye (such as Hoechst 33258, Hoechst 33342, Hoechst 34580), a CYBR® dye (such as CYBR® Gold, CYBR® Green, CYBR® Safe), and EVAGREEN®. In some embodiments, the dye is a cyanine dye. Examples of cyanine dyes are listed in Table 1 below. In some embodiments, use of a dye in combination with a biosensor loaded with oppositely charged biopolymers enables direct signal visualization and amplification on the BLI platform and provides a more rapid and efficient detection capability compared to a sandwich assay, for instance using a first antibody that binds to ssDNA, and a secondary antibody with a signal development element (such as HRP) that binds to the first antibody.

TABLE 1
Example DNA-binding cyanine dyes

| | | | In: DNA Presence (nm) | | |
|---|---|---|---|---|---|
| [symbol] | MW | Chemical Structure | [symbol] | [symbol] | [symbol] |
| 1 | 650.29 | 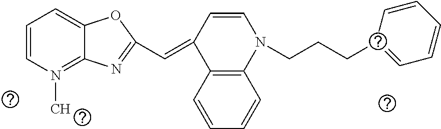 | 531 | 533 | 547 |
| 2 | 825.31 | 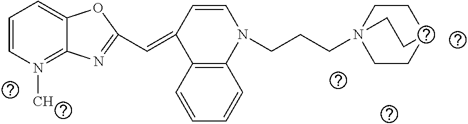 | 531 | 533 | 548 |
| 3 | 839.33 | 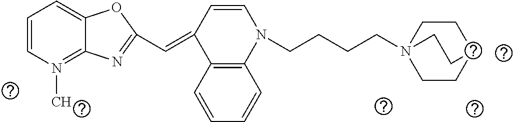 | 532 | 533 | 547 |
| 4 | 833.36 | 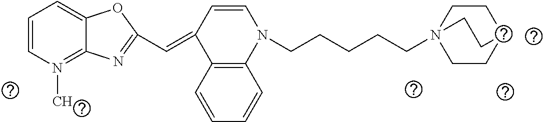 | 531 | 533 | 549 |
| 5 | 543.36 | 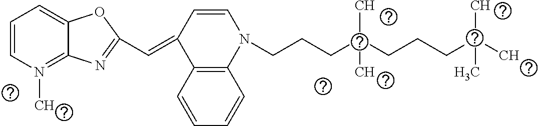 | 531 | 533 | 546 |
| 6 | 857.39 | 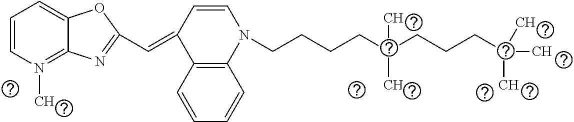 | 531 | 533 | 547 |
| 7 | 871.42 | 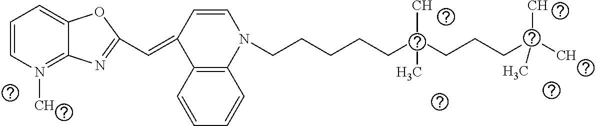 | 532 | 534 | 548 |
| 8 | 951.54 | 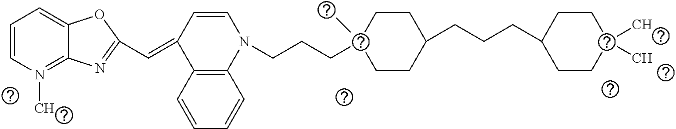 | 531 | 533 | 546 |
| 9 | 963.37 | 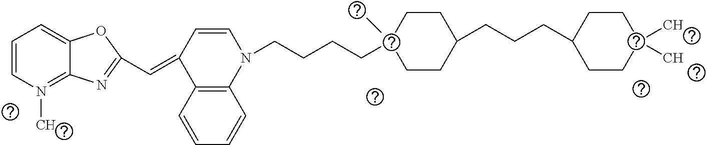 | 532 | 534 | 547 |
| 10 | 979.60 | 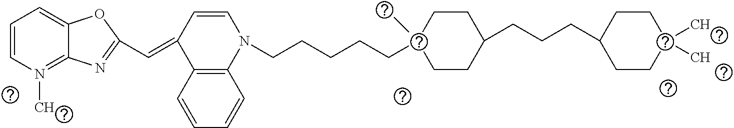 | 537 | 534 | 546 |
| 11 | 1300.67 | 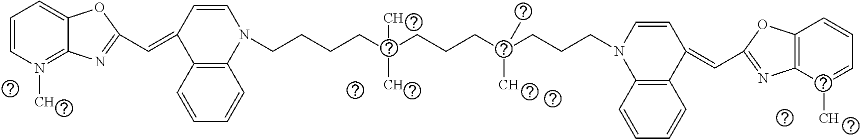 | 530 | 532 | 547 |
| 12 | 1408.20 | 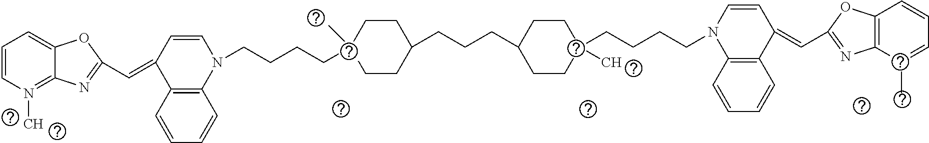 | 532 | 534 | 547 |
| POPO-3 iodide | 1222.61 | 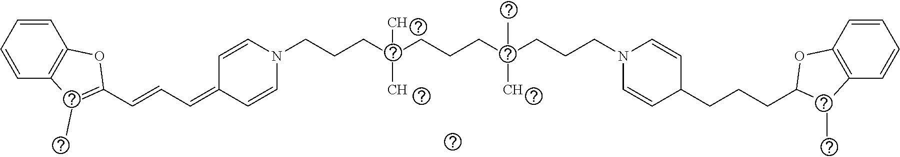 [SMILES: Cc1c(/C=C/C=C2C=CN(CCCC(C)(C)CCCC(C)(C)CCCN3C=CC(CCCC4Oc5ccccc5C4C)C=C3)C=C2)oc2ccccc12] | 533 | 534 | 570 |
| YOYO-1 iodide | 1270.64 | 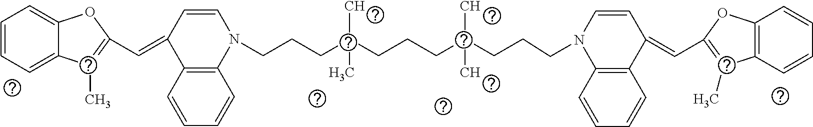 | 491 | 488 | 509 |

MW = molecular weight; λ_{abs}, λₑₓ, λₑₘ - maximum wavelength of absorption, excitation, and emission spectra. Zarkov et al. 2013 Molecular Imaging 12.2:7290-2012. [symbol]
indicates data missing or illegible when filed

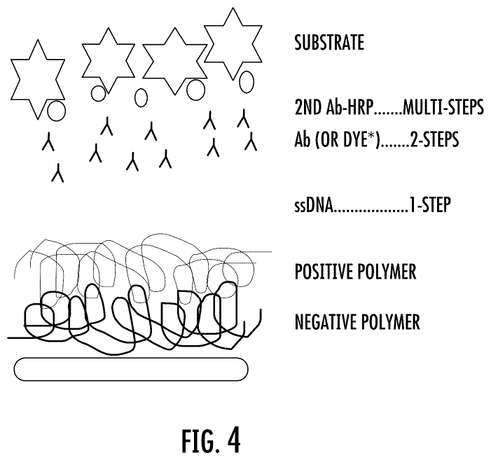

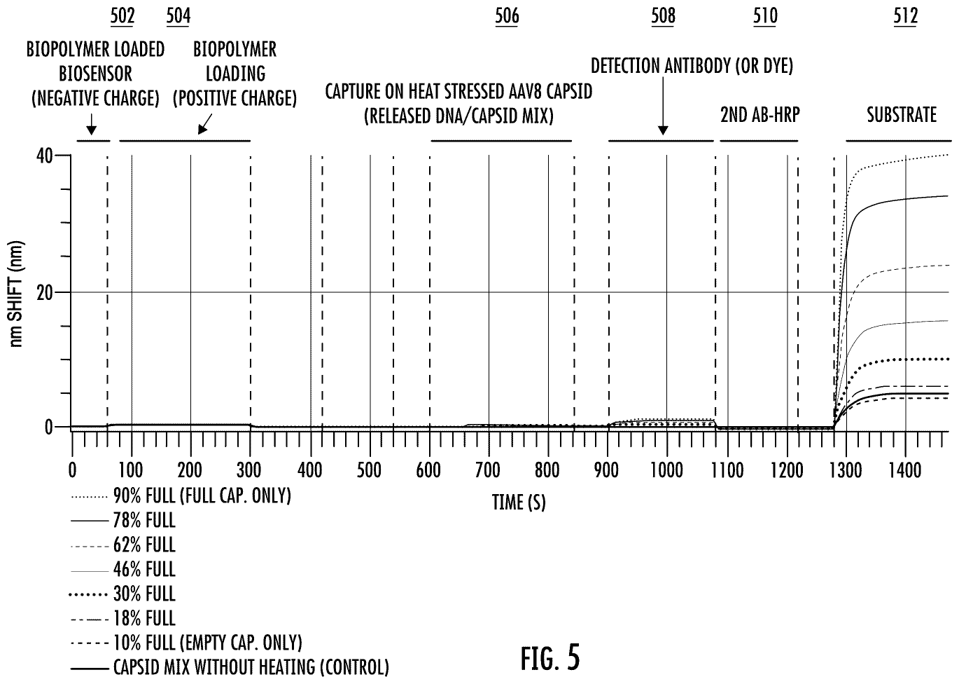

In some embodiments, an enzyme (such as HRP, AP, 0-galactosidase, acetylcholinesterase, catalase)-linked antibody is used to quantitate polynucleotides bound to the BLI biosensor, where the enzyme catalyzes the conversion of a substrate for the detection of polynucleotides bound to the BLI biosensor. For example, as depicted in an example process in FIGS. 4 and 5, the polynucleotides (such as ssDNA) can be contacted with a primary antibody (detection antibody) or dye that binds polynucleotides to allow for binding (phase 508 of FIG. 5). The polynucleotides/primary antibody (or dye) complex can then be contacted with an enzyme (such as HRP)-linked secondary antibody to allow for binding (phase 510 of FIG. 5). The polynucleotides/primary antibody (or dye)/enzyme-linked secondary antibody complex can then be contacted with a substrate. In an embodiment, the enzyme initiates polymerization of the substrate resulting in formation of non-soluble precipitates on the surface of the BLI biosensor. The precipitates introduce thickness changes on the surface of the BLI biosensor that correspond to signal (nm) shift (phase 512 of FIG. 5). In another embodiments, the enzyme catalyzes the conversion of the substrate for the detection of the polynucleotides bound to the BLI biosensor.

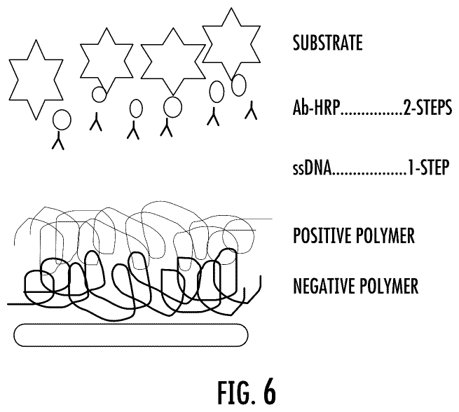

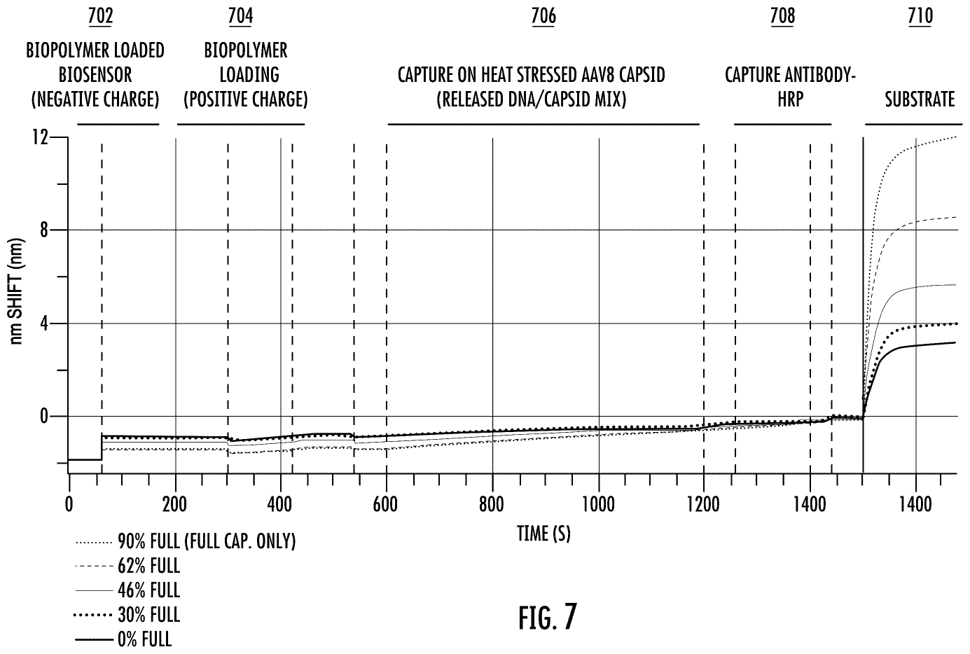

As another example, as depicted in an example process in FIGS. 6 and 7, the polynucleotides (such as ssDNA) can be contacted with an enzyme (such as HRP)-linked capture antibody that binds polynucleotides to allow for binding (phase 708 of FIG. 7). The polynucleotides/enzyme-linked antibody complex can then be contacted with a substrate. In an embodiment, the enzyme initiates polymerization of the substrate resulting in formation of non-soluble precipitates on the surface of the BLI biosensor. The precipitates introduce thickness changes on the surface of the BLI biosensor that correspond to signal (nm) shift (phase 710 of FIG. 7). In another embodiment, the enzyme catalyzes the conversion of the substrate for the detection of the polynucleotides bound to the BLI biosensor.

In the example processes shown in FIGS. 4-7, BLI can be used to measure the signal/wavelength/spectral shift (nm) over time to detect signals generated by the enzyme and the substrate, with a positive shift indicating polynucleotide binding to the biosensor.

In some embodiments, the method further comprises determining a ratio of empty and full viral capsids in a sample. The empty/full capsid ratio can be calculated based on one or more measurements in a subject/unknown sample and/or a reference sample. A “subject/unknown sample” as used herein refers to a sample with an unknown viral titer and/or an unknown empty and full viral capsid ratio. A “reference sample” as used herein refers to a sample with a known viral titer and/or a known empty and full viral capsid ratio. For example, the empty/full capsid ratio in subject samples can be calculated based at least partially on the amount of polynucleotides bound to the biosensor (indicative of the amount of polynucleotides contained in the viral capsids in the sample) in a reference sample and/or a subject sample. For example, a calibration curve can be generated based on the signals or the binding kinetics of reference samples obtained according to the methods provided herein, and the empty/full viral capsid ratio in subject samples can be determined using the calibration curve.

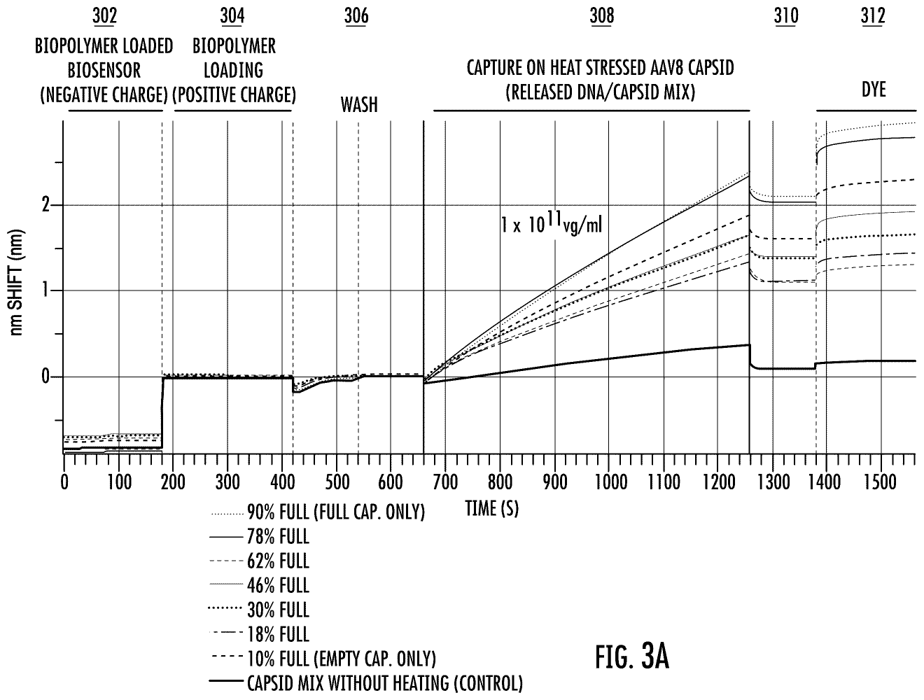

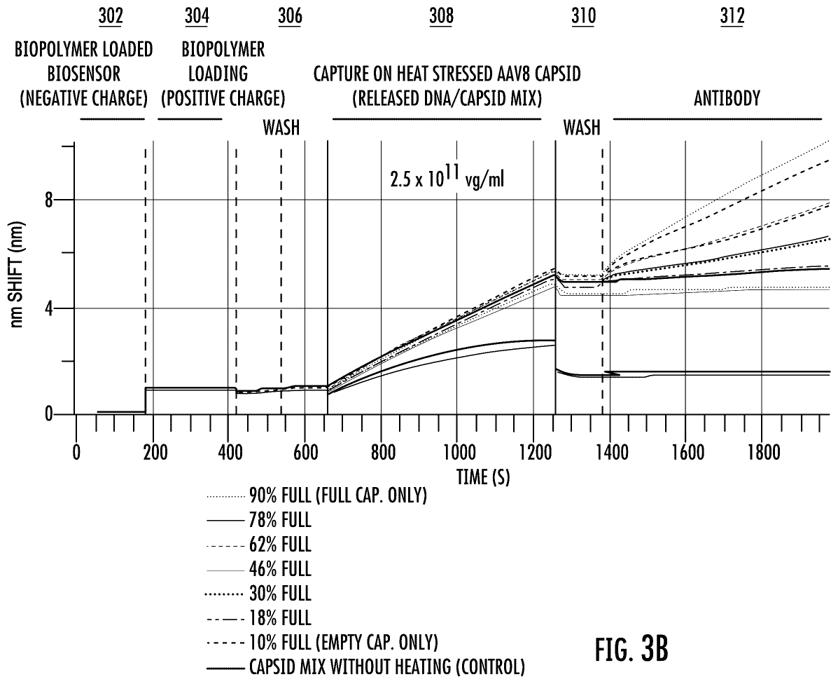

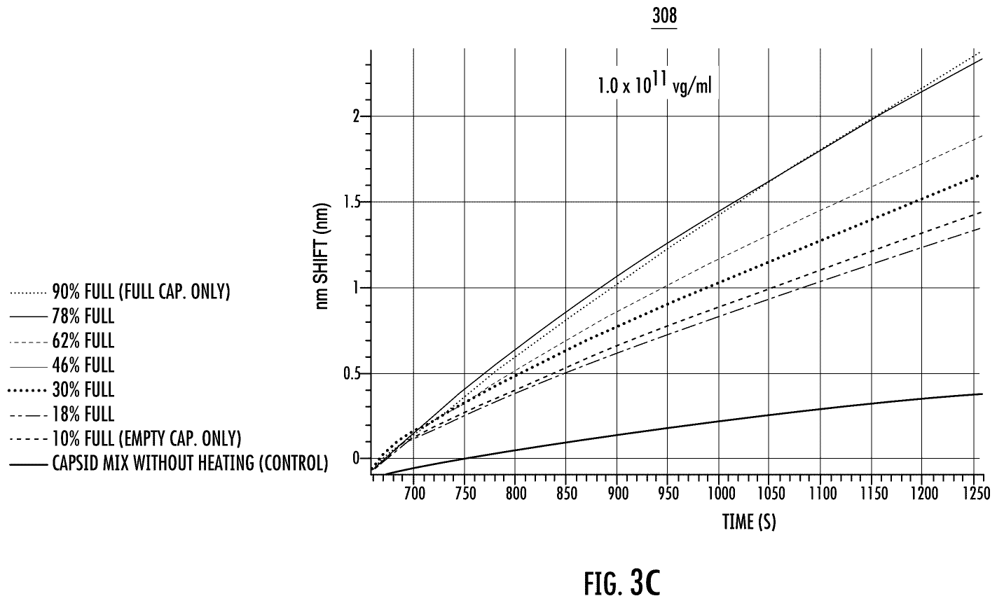

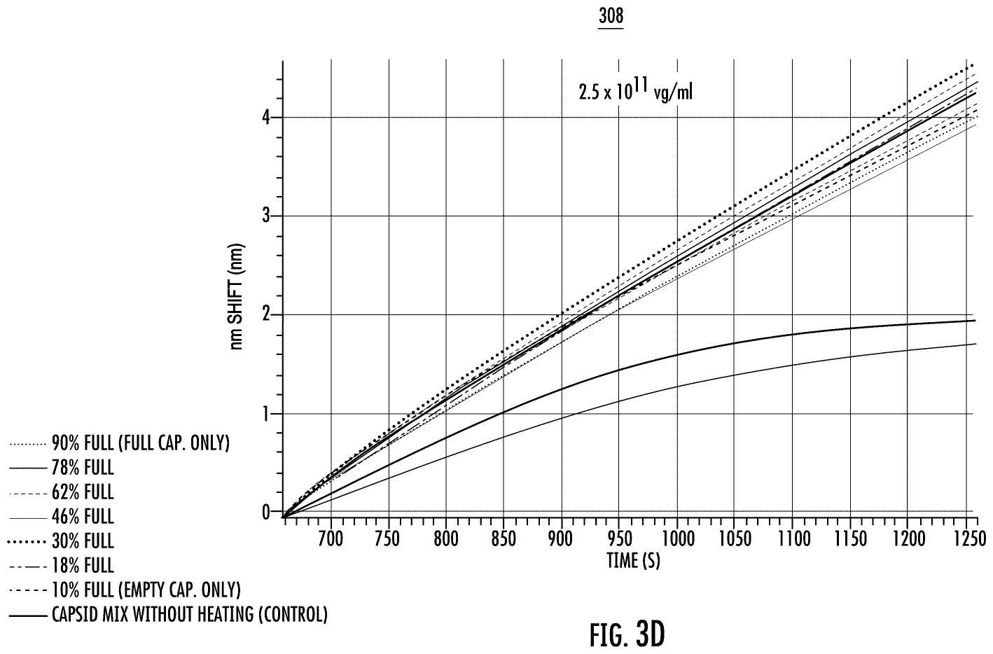

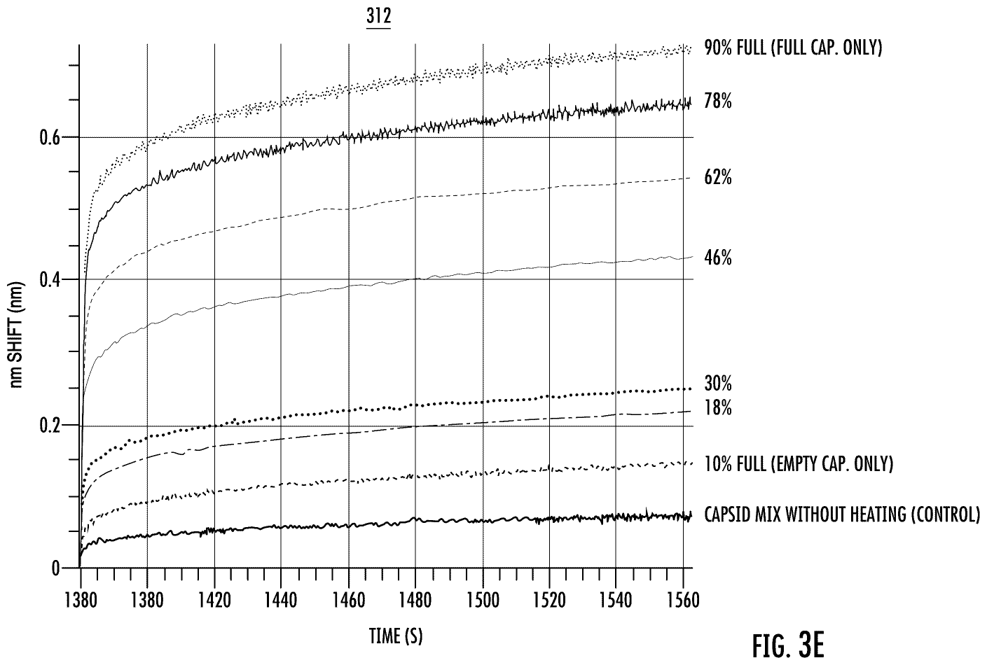

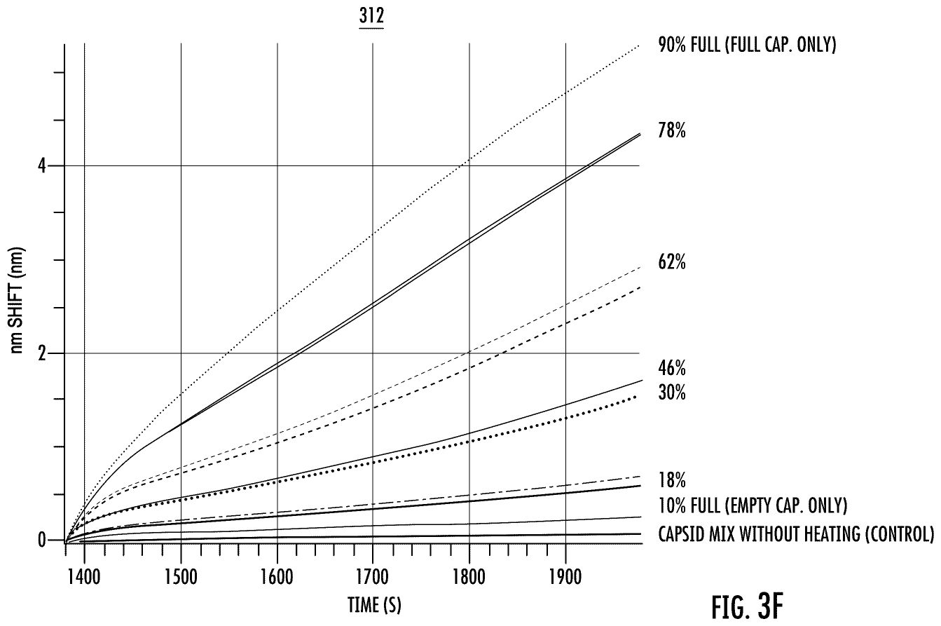

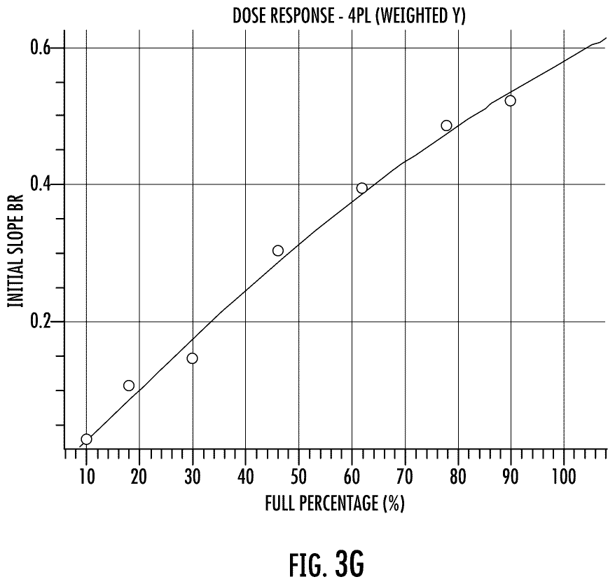

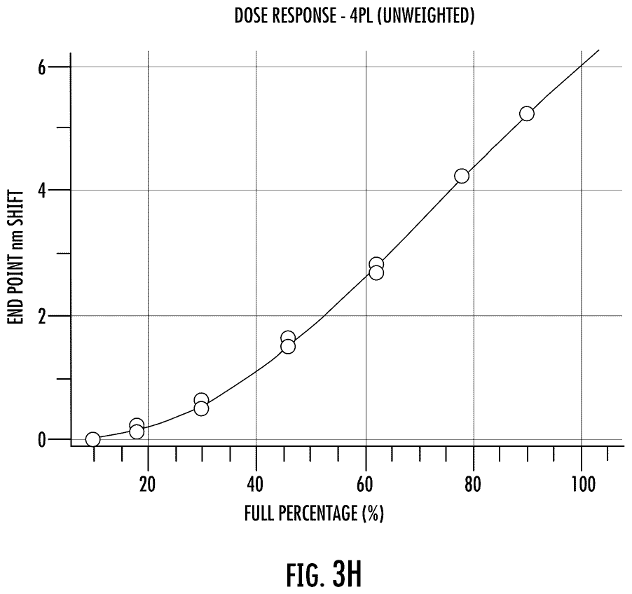

Further, as shown FIGS. 3A, 3B, 3E, 3F 3G, and 3H, in the signal amplification phase 312, samples with higher full capsid ratio can have greater signal changes (increases) as compared to samples with higher empty capsid ratio because a sample having a higher full capsid ratio has higher DNA concentrations as compared to a sample having a higher empty capsid ratio. In some such embodiments, the empty/full capsid ratio can be determined based at least partially on the binding kinetics of the biosensor towards the polynucleotides in a reference sample and/or a subject sample.

In some embodiments, the method further comprises, prior to the step of separating the polynucleotides from one or more viral capsids, contacting the intact viral capsids with the biopolymer-based biosensor, wherein upon contacting with the biosensor, the intact viral capsids bind to the positively charged biopolymer of the biosensor. Viral capsids are negatively charged, and therefore bind to the positively charged biopolymer layer and generate a set of signals. This step can be useful for directly quantifying the amount of viral capsids in a sample for titer determination.

In some embodiments, the method further comprises, prior to the step of separating the polynucleotides from one or more viral capsids, contacting the viral capsids with an affinity biosensor to bind the viral capsids with the affinity biosensor. Viral capsids can be isolated or purified from a crude sample (containing cells, particles, debris, or compounds other than viral capsids) using the affinity biosensor. An affinity biosensor used herein can be an antibody-based affinity biosensor. For example, an AAVX biosensor can be used to isolate or purify AAV viral capsids from a crude sample. For example, the method provided herein can include contacting the viral capsids (such as AAV capsids) with a first biosensor (affinity biosensor, such as AAVX biosensor) to isolate and/or purify the viral capsids from a crude sample, followed by subjecting the viral capsids bound to the first biosensor to a separation process to separate the polynucleotides from the viral capsids, contacting the polynucleotides with a second biosensor (such as biopolymer-based biosensor) such that the polynucleotides are bound to the second biosensor, contacting the polynucleotides bound to the second biosensor with signal development elements (such as a dye or an antibody) to generate signals indicative of an amount of the polynucleotides bound to the second biosensor, and determining a ratio of empty and full viral capsids based at least partially on the amount of viral capsids bound to the first biosensor, the amount of polynucleotides bound to the second biosensor and/or binding kinetics of the second biosensor to the polynucleotides.

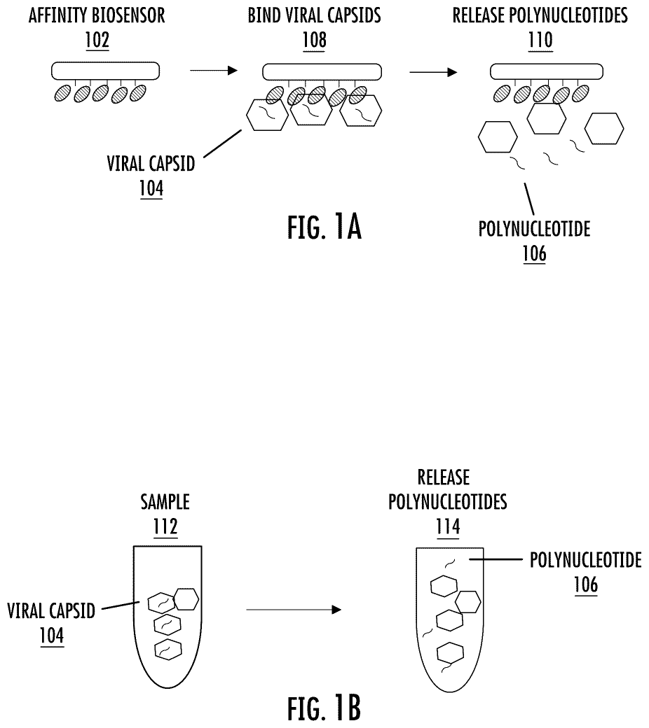

FIGS. 1A and 1B schematically depict a method of separating viral capsids and polynucleotides (such as ssDNA) contained therein. FIG. 1A depicts an embodiment of purifying viral capsids 104 from a crude sample, which can contain cells, particles, debris, or compounds other than viral capsids, by contacting the viral capsids with an affinity biosensor 102, allowing the viral capsids to bind to the affinity biosensor, as shown in step 108. This step can be followed by step 110, which includes applying heat stress to the viral capsids 104 and releasing the polynucleotides 106 (such as ssDNA) from the viral capsids having been bound to the biosensor. FIG. 1B depicts separating viral capsids 104 and polynucleotides 106 (such as ssDNA) in a sample 112, such as a purified sample, that is substantially free of compounds other than viral capsids, by applying heat stress in step 114.

In some embodiments, the viral capsids in the sample to be analyzed are AAV (including recombinant AAV) capsids. Samples can contain a range of titer of AAV. In some embodiments, the titer of AAV in the composition is 1×10¹⁰ to 1×10¹⁴ viral genomes/mL (vg/mL), such as 1×10¹⁰ to 1×10¹³ vg/mL, 1×10¹⁰ to 1×10¹² vg/mL, 1×10¹¹ to 1×10¹⁴ vg/mL, or 1×10¹¹ to 1×10¹³ vg/mL. In a specific embodiment, the titer of AAV in the composition is 3×10¹⁰ to 5×10¹¹ viral genomes/mL (vg/mL). In some embodiments, the empty/full capsid ratio in the sample is 0-10%, 10-20%, 20-30%, 30-40%, 40-50%, 50-60%, 60-70%, 70-80%, 80-90%, or 90-10%, such as 10%, 20%, 30%, 40%, 50%, 60%, 70%, 80%, 90%, or 100%. In some embodiments, the AAV particles in the sample have been purified using a purification step, such as those provided herein. In some embodiments, the AAV particles in the sample have not been purified.

The full and empty AAV capsids in any given sample may belong to any serotype, including natural and recombinant serotypes, including chimeric (mixed) and novel serotypes. It will be understood that capsids of different serotypes have different charge properties, including different levels of charge distinction between empty AAV capsids and full capsids, or among different recombinant constructs within a serotype. In some embodiments, the viral particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAV13 capsid, an AAV14 capsid, an AAV15 capsid, an AAV16 capsid, an AAVrh20 capsid, an AAV.rh39 capsid, an AAV.Rh74 capsid, an AAV.RHM4-1 capsid, an AAV.hu37 capsid, an AAV.Anc80 capsid, an AAV.Anc80L65 capsid, an AAV.PHP.B capsid, an AAV2.5 capsid, an AAV2tYF capsid, an AAV3B capsid, an AAV.LK03 capsid, an AAV.HSC1 capsid, an AAV.HSC2 capsid, an AAV.HSC3 capsid, an AAV.HSC4 capsid, an AAV.HSC5 capsid, an AAV.HSC6 capsid, an AAV.HSC7 capsid, an AAV.HSC8 capsid, an AAV.HSC9 capsid, an AAV.HSC10 capsid, an AAV.HSC11 capsid, an AAV.HSC12 capsid, an AAV.HSC13 capsid, an AAV.HSC14 capsid, an AAV.HSC15 capsid, an AAV.HSC16 capsid, an AAV2R471A capsid, an AAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid. In some specific embodiments, the AAV particles are AAV2 particles. In some embodiments, the AAV genome is 2500 bases to 5500 bases, 3000 bases and 5500 bases, 3500 bases and 5500 bases, 4000 bases and 5500 bases, 4500 bases and 5500 bases, 5000 bases and 5500 bases, 2500 bases and 5000 bases, 3000 bases and 5000 bases, 3500 bases and 5000 bases, 4000 bases and 5000 bases, 4500 bases and 500 bases, 2500 bases and 4500 bases, 3000 bases and 4500 bases, 3500 bases and 4500 bases, 4000 bases and 4500, 2500 bases and 4000 bases, 3000 bases and 4000 bases, 3500 bases and 4000 bases, 2500 bases and 3500 bases, 3000 bases and 3500 bases, or 2500 bases and 3000 bases. In some embodiments, the recombinant viral particles comprise a self-complementary AAV (scAAV) genome.

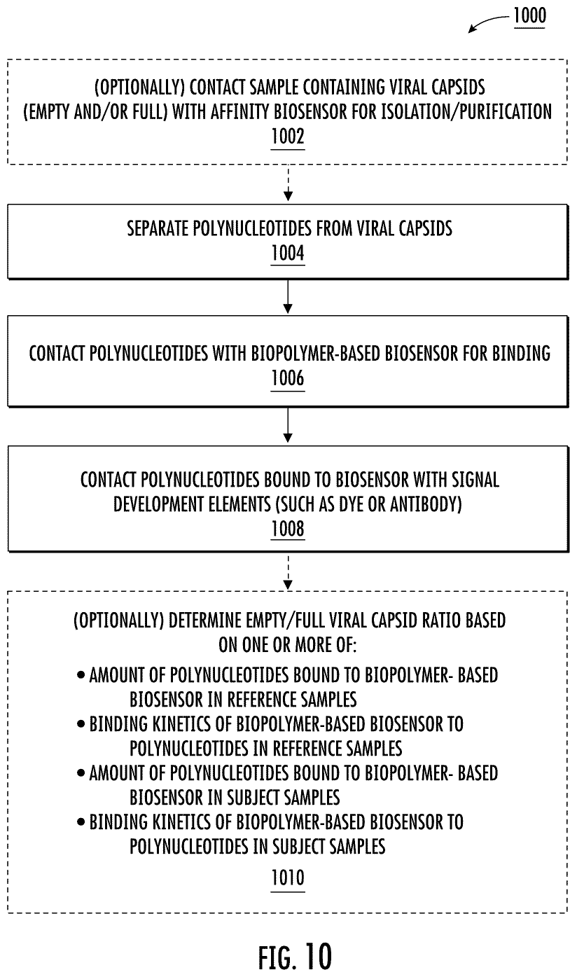

FIG. 10 depicts an example method 1000 of quantitating a ratio of viral capsids containing polynucleotides in a sample, according to embodiments of the present disclosure. The process 1000 begins from step 1004 by separating polynucleotides from viral capsids, for example by applying heat stress to the viral capsids. Pure samples, substantially free of compounds other than viral capsids, can start at step 1004. In analyzing crude samples, comprising cells, debris, particles, or compounds other than viral capsids, the process 1000 can begin by purifying or isolating the viral capsids. At step 1002, the process includes contacting the sample containing the viral capsids with an affinity biosensor such that the viral capsids bind to the affinity biosensor and can be isolated or purified. Process 1000 can then continue to step 1004 described above. Process 1000 continues to step 1006, to contact the polynucleotides separated from the viral capsids with the biosensor such that the polynucleotides bind to the biosensor. Process 1000 continues to step 1008, to contact the polynucleotides bound to the biosensor with signal development elements (such as dye, for example a cyanine dye, or an antibody, such as a ssDNA specific antibody) to generate signals indicative of the amount of the polynucleotides bound to the biosensor. The signals can be indicative of wavelength/spectral/nm shift measured using bio-layer interferometry (BLI). A positive shift can indicate binding of the polynucleotides to the biosensor. Process 1000 can optionally continue to step 1010, to determine the empty and full viral capsid ratios based on one or more of the following parameters: the amount of polynucleotides bound to the charged biosensor, as indicated by signals generated by binding of the signal development elements to the polynucleotides bound to the biosensor; and the binding kinetics of the biosensor to the polynucleotides, which indicate the empty and full viral capsid ratios in the sample. The empty/full viral capsid ratio in subject samples (in which the empty/full viral capsid ratio is unknown) can be determined using a calibration curve generated based on the signals or the binding kinetics of reference samples (in which the empty/full capsid ratio is known) obtained using the same method disclosed here.

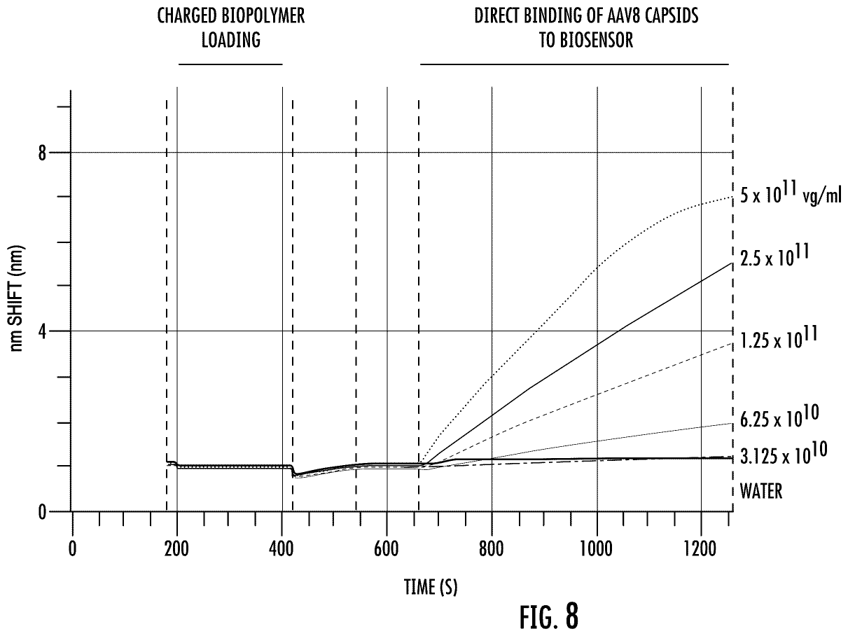

Also provided herein is a method for quantitating the amount of viral capsids in a sample. Viral capsids are negatively charged, and therefore directly bind to the positively charged biopolymer layer of a biosensor. This method can be useful for directly quantifying the amount of viral capsids in a sample for titer determination. The method includes the steps of: contacting the viral capsids, contained in samples with different titers but the same empty vs full viral capsid ratio, with a biopolymer based biosensor provided herein to bind viral capsids to a positively charged biopolymer of the biosensor and generate signals indicative of binding amount of the viral capsids to the biosensor and quantitating amount of viral capsid in a sample. The signals can be indicative of wavelength/spectral/nm shift measured using bio-layer interferometry (BLI). A positive wavelength/spectral/nm shift can indicate binding of the one or more viral capsids to the biosensor. For example, as shown in FIG. 8, the binding signal level of viral capsids is correlated with capsid titers with a lower capsid titer in a sample showing slower and lower binding signal compared to a higher capsid titer in a sample showing a faster and higher binding signal. The viral titer in subject samples can be determined using a calibration curve generated based on the signals of reference samples obtained using the same method disclosed here.

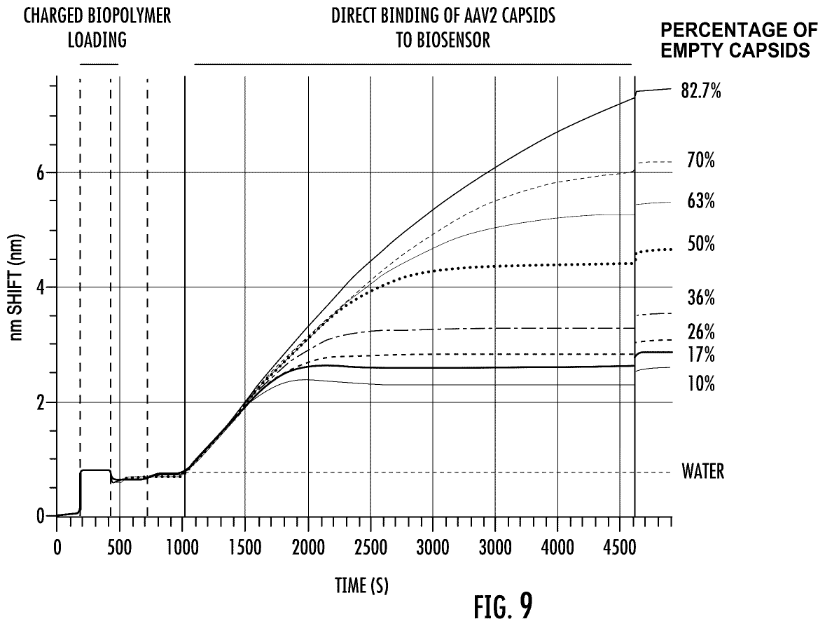

Also provided herein is a method for directly quantitating empty vs full ratio of viral capsids (such as AAV viral capsids) in a sample. Viral capsids are negatively charged, and therefore directly bind to the positively charged biopolymer layer of a biosensor. The method includes the steps of: contacting the viral capsids, contained in samples with the same titer, but different empty vs full viral capsid ratios, with a biopolymer-based biosensor provided herein to bind viral capsids to a positively charged biopolymer of the biosensor and generate signals indicative of binding of the viral capsids to the biosensor; and quantitating of a ratio of viral capsids (such as AAV viral capsids) containing polynucleotide. For example, as shown in FIG. 9, the binding of viral capsids (empty and full) to the biosensor can reach saturation quicker in samples with a lower empty capsid ratio as compared to samples with a higher empty capsid ratio because a full capsid with a DNA core can have higher negative charges as compared to an empty capsid without a DNA core. The empty/full viral capsid ratio in subject samples can be determined using a calibration curve generated based on the binding kinetics of reference samples obtained using the same method disclosed here. In some such embodiments, the empty/full capsid ratio cam be determined based at least partially on the binding kinetics of the biosensor towards the viral capsids. Accordingly, the method provided herein may further comprise determining a ratio of empty and full viral capsids based at least partially on the binding kinetics of the biosensor to the viral capsids. In certain embodiments, the signals (including binding kinetics) are measured using BLI.

The method for quantitating the amount of viral capsids or the method for directly quantitating empty vs full ratio of viral capsids described herein can be combined with the method for quantitating the amount of one or more polynucleotides in a sample. For example, the method provided herein that includes binding viral capsids to a biosensor can further include subjecting one or more viral capsids bound to the biosensor to a separation process to separate the polynucleotides from the one or more viral capsids, contacting the polynucleotides with the biosensor to bind the polynucleotides to the positively charged biopolymer of the biosensor, and contacting the polynucleotides bound to the biosensor with one or more signal development elements to generate another set of signals indicative of an amount of the polynucleotides bound to the biosensor.

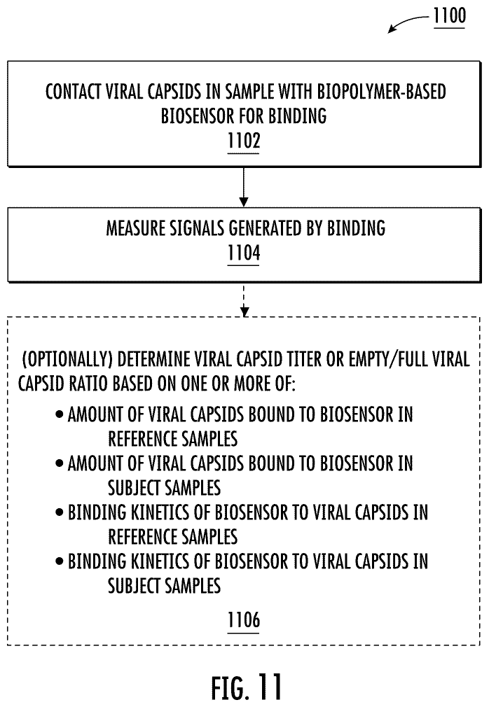

FIG. 11 depicts an example method 1100 of quantitating an amount of viral capsids in a sample or empty vs full ratio of capsids in a sample, according to embodiments of the present disclosure. The process 1100 begins from step 1102 by contacting the viral capsids contained in the sample with the biosensor such that the viral capsids bind to the biosensor. Process 1100 continues to step 1104, to measure signals generated by binding of the viral capsids to the biosensor. The signals can be indicative of wavelength/spectral/nm shift measured using bio-layer interferometry (BLI). A positive shift can indicate binding of the one or more viral capsids to the biosensor. Process 1100 can optionally continue to step 1106, to determine the viral capsid titer and/or empty and full viral capsid ratios based on one or both of the following parameters: the amount of viral capsids bound to the biosensor; and the binding kinetics of the biosensor to the viral capsids.

In further embodiments, the method 1100 can further include the steps of subjecting the one or more viral capsids bound to the biosensor to a separation process to separate the polynucleotides from the one or more viral capsids, contacting the polynucleotides with the biosensor to bind the polynucleotides to the positively charged biopolymer of the biosensor, and contacting the polynucleotides bound to the biosensor with signal development elements to generate signals indicative of an amount of the polynucleotides bound to the biosensor. The separation (polynucleotide release), polynucleotide capture, and signal amplification processes described in elsewhere in the present disclosure can be used.

In some embodiments, the biosensor comprises an optical fiber having a proximal end portion and a distal end portion, the proximal end portion configured to receive light from a light source and configured to deliver reflected light to a detector. The distal end portion configured to have analytes bind thereto such that light reflected from the distal end portion is phase shifted based on a thickness of analytes bound to the distal end portion. In some embodiments, the biosensor further comprises an optical resonator at a distal end portion of the optical fiber, the optical resonator including a first reflective surface and a second reflective surface, the first reflective surface configured to reflect light with a first phase and the second reflective surface configured to reflect light with a second phase which is phase shifted based on a thickness of analytes bound to the optical resonator.

In some embodiments of the methods provided herein, the signals such as those generated by binding of the signal development elements to the polynucleotides and/or those generated by binding of the viral capsids to the biosensor are measured by a detector. The signals can be detected based on any label-free technique for detecting a change in a property of a sensor surface, such as BLI, Surface Plasmon Resonance (SPR), Surface Acoustic Wave (SAW), Quartz Crystal Microbalance (QCM), and Reflectometric Interference Spectroscopy (RIfS). In specific embodiments, the signals are measured by an interferometer. The interferometer can comprise the biosensor, and can constitute a BLI sensor. In some embodiments, the interferometer further comprises: a first optical waveguide configured to receive light from a light source; a second optical waveguide configured to deliver reflected light to a detector; and an optical coupler spatially separates a distal portion of the first optical waveguide from a distal portion of the second optical waveguide, wherein the biosensor is attached to the optical coupler. In some embodiments, the interferometer further comprises a light source that is in optical communication with the first optical waveguide and configured to provide light to the first optical waveguide. In some embodiments, the interferometer further comprises a detector configured to receive light from the second optical waveguide. In some embodiments, the first optical waveguide and the second optical waveguide are disposed in a fiber optic bundle.

For example, an interferometer can include a light source, an optical assembly, and a detector unit. The bio-layer interferometry (BLI) sensor or optical assembly functions as a sensing element or detector tip to detect analytes attached to an end thereof. The detector unit detects interference signals produced by interfering light waves reflected from the optical assembly. The light source directs light into the optical assembly, which is reflected back to the detector unit through an optical coupling assembly. The coupling assembly includes a first optical waveguide or fiber that extends from the light source to the optical assembly, a second optical waveguide or fiber which carry reflected light from the optical assembly to the detector, and an optical coupler which optically couples the first optical waveguide and the second optical waveguide. In some embodiments, the coupling assembly includes a lens system constructed to focus a light beam on an upper surface of the optical assembly and to direct reflected interfering light from the optical assembly to the detector.

The light source can be a white light source, such as a light emitting diode (LED), that produces light over a broad spectrum, e.g., 400 nm or less to 700 nm or greater, typically over a spectral range of at least 100 nm. In some embodiments, the light source can be a plurality of sources each having a different characteristic wavelength, such as LEDs designed for light emission at different selected wavelengths in the visible light range. The same function can be achieved by a single light source, such as, white light source, with suitable filters for directing light with different selected wavelengths onto the optical assembly.

The detector may be a spectrometer, such as charge-coupled device (CCD), capable of recording the spectrum of the reflected interfering light from the optical assembly. In some embodiments, where the light source operates to direct different selected wavelengths onto the optical assembly, the detector may be a simple photodetector for recording light intensity at each of the different irradiating wavelengths. In certain embodiments, the detector may include one or more filters which allows detection of light intensity, for instance from a white-light source, at each of a plurality of selected wavelengths of the interference reflectance wave.

The first optical waveguide and/or the second optical waveguide may be in the form of a fiber optic bundle (FOB). As shown, the first optical waveguide includes several fiber optic elements surrounding a single fiber optic element of the second optical waveguide. This arrangement separates delivery of light from the light source from delivery of the reflected light from the optical assembly to the detector. It will be appreciated that other arrangements of the first optical waveguide and the second optical waveguide may allow for spatial separation of the light from the light source and the reflected light from the optical assembly. The separation of the light from the light source and the reflected light from the optical assembly may improve a signal to noise ratio (SNR) of the apparatus. In some embodiments, the first optical waveguide is a single fiber and the second fiber optical waveguide is formed of a plurality of fibers.

The distal tip of the fiber optic bundle can be aligned with a proximal end portion of the optical fiber when the BLI sensor is attached to the optical coupler. The BLI sensor may be fixedly attached to the optical coupler to align and maintain a position of the proximal end portion with respect to the tip.

The BLI sensor can include the optical fiber having a proximal end and a distal end. The proximal end and/or the distal end of the optical fiber may be polished ends. The BLI sensor can have an optical resonator having a first reflecting surface and a second reflecting surface distal of the first reflecting surface. The optical fiber is substantially transparent between the proximal end and distal end thereof. The optical resonator may be transparent between the first and second reflecting surfaces. The distance between the first and second reflecting surfaces defines a thickness of the optical resonator. The thickness of the optical resonator may be in a range of 50 nm to 5,000 nm, such as between 400 nm and 1,000 nm.

The second reflecting surface is formed of a layer of analyte-binding molecules which are effective to bind analyte molecules specifically and with high affinity. That is, the analyte and anti-analyte molecules are opposite members of a binding pair which can include, without limitations, antigen-antibody pairs, complementary nucleic acids, and receptor-binding agent pairs. In specific embodiments, the analyte and anti-analyte molecules are oppositely charged. For example, the analyte and anti-analyte molecules are negatively and positively charged, respectively.

The index of refraction of the optical fiber may be similar to that of the second reflecting surface so that light reflected from the second reflecting surface occurs predominantly from the layer formed by the analyte-binding molecules, rather than from the interface between the optical fiber and the analyte-binding molecules. Similarly, as analyte molecules bind to distal end portion of the optical assembly, light reflected from the distal end portion of the assembly occurs predominantly from the layer formed by the analyte-binding molecules and bound analyte, rather than from the interface region.

The first reflecting surface of the optical assembly is formed as a layer of transparent material having an index of refraction that is substantially different from that of the optical fiber, such that this layer functions to reflect a portion of the light directed onto the optical assembly.

The thickness of an analyte-binding layer disposed in the distal end portion of the optical element may be designed to optimize the overall sensitivity based on specific hardware and optical components. Conventional immobilization chemistries are used in chemically, such as covalently, attaching a layer of analyte-binding molecules to the lower surface of the optical element. For example, a variety of bifunctional reagents containing a siloxane group for chemical attachment to SiO₂, and a hydroxyl, amine, carboxyl or other reaction group for attachment of biological molecules, such as proteins (such as antigens, antibodies), or nucleic acids. It is also well known to etch or otherwise treat glass or glass surfaces to increase the density of hydroxyl groups by which analyte-binding molecules can be bound. Where the optical fiber is formed of a polymer, such as polystyrene, a variety of methods are available for exposing available chemically active surface groups, such as amine, hydroxyl, and carboxyl groups.

In certain embodiments, the analyte-binding layer is formed under conditions in which a distal end surface of the optical fiber is densely coated, so that binding of analyte molecules to the layer forces a change in the thickness of the layer, rather than filling in the layer. The analyte-binding layer can be either a monolayer or a multi-layer matrix.

The measurement of the presence, concentration, and/or binding rate of analyte to the optical assembly is enabled by the interference of reflected light beams from the two reflecting surfaces in the optical assembly. Specifically, as analyte molecules attach to or detach from the surface, the average thickness of the first reflecting layer changes accordingly. Because the thickness of all other layers remains the same, the interference wave formed by the light waves reflected from the two surfaces is phase shifted in accordance with this thickness change.

Assuming that there are two reflected beams, the first beam is reflected from the first reflecting surface and the second beam is reflected from the analyte-binding molecules and bound analyte and the surrounding medium at the second reflecting surface. The conversion of the phase shifting to a thickness change of the bound analytes is well known in the art.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

### EXAMPLES

### Example 1: Binding Kinetics of Adeno-Associated Virus (AAV) Polynucleotides to a Bio-Layer Interferometry (BLI) Biosensor and Associated Interferometry Signals

This example relates to binding kinetics of single-stranded DNA (ssDNA) isolated from AAV8 capsids to the bio-layer interferometer (BLI) biosensor in samples with varied empty/full capsid ratios. Four methods were used to detect ssDNA: 1) detection using a DNA-binding cyanine dye (FIG. 3A); 2) detection using a ssDNA specific antibody (FIG. 3B); 3) detection using a ssDNA-binding primary antibody or dye, a HRP-linked secondary antibody, and a substrate (FIGS. 4 and 5); and 4) detection using a HRP-linked detection antibody and a substrate (FIGS. 6 and 7).

In the first and second methods, the BLI biosensor loaded with negatively charged biopolymer was equilibrated (phase 302 of FIGS. 3A and 3B), and then loaded with polyethylenimine (PEI) (phase 304 of FIGS. 3A and 3B), with appropriate washes (phase 306 of FIGS. 3A and 3B). Heat stress was applied to samples each containing AAV8 with the full capsid ratio of 10%, 18%, 30%, 46%, 62%, 78%, and 90%, respectively, to allow ssDNA contained in the viral capsids to be released and bound to the biosensor (phase 308 of FIGS. 3A and 3B). In FIG. 3A, the sample contained 1×10¹¹ vg/ml of AAV8. In FIG. 3B, the sample contained 2.5×10¹¹ vg/ml of AAV8. BLI was used to measure the signal shift (nm) overtime for the heat-stressed samples that were bound to the BLI biosensor, with a positive shift indicating binding partially from viral capsid and partially from polynucleotide to the biosensor. When the signal shift reached certain nm shift (in other words, enough capsid and/or polynucleotide bound), a wash was performed (phase 310 of FIGS. 3A and 3B) and a dye or an antibody was added to allow binding to the ssDNA bound to the biosensor. In FIG. 3A, a DNA-binding cyanine dye, was added to allow binding to the ssDNA bound to the BLI biosensor (phase 312). In FIG. 3B, a ssDNA specific antibody was added to allow binding to the ssDNA bound to the BLI biosensor (phase 312).

FIGS. 3A and 3B depict the overview of the signal change over the process comprising the negatively charged biopolymer preloaded phase 302, the positively charged biopolymer loading phase 304, the polynucleotide/viral capsid capture phase 308, the signal amplification phase 312, and washes in between (306 and 310), described above. As shown in FIG. 3C (polynucleotide capture phase 308 of FIG. 3A) and 3D (polynucleotide capture phase 308 of FIG. 3B), increase of nm shift indicates the binding of capsid and polynucleotide in samples to positively charged biopolymer layer. As shown FIG. 3E (signal amplification phase 312 with a dye), 3F (signal amplification phase 312 with an antibody), FIG. 3F (dye-amplified signals in AAV8 samples with different full capsid ratios), and FIG. 3G (antibody-amplified signals in AAV8 samples with different full capsid ratios), in the signal amplification phase 312, samples with higher full capsid ratio showed greater signal changes as compared to samples with lower full capsid ratio. This can be explained by the higher full capsid ratio having higher DNA concentrations in a sample as compared to a sample having a higher empty capsid ratio.

The third method is depicted in FIGS. 4 and 5. In the third method, the BLI biosensor loaded with negatively charged biopolymer was equilibrated (phase 502 of FIG. 5), and then was loaded with polyethylenimine (PEI) (phase 504 of FIG. 5), with appropriate washes. Heat stress was applied to samples each containing AAV8 with the full capsid ratio of 10%, 18%, 30%, 46%, 62%, 78%, and 90%, respectively, to allow ssDNA contained in the viral capsids to be released and bound to the biosensor (phase 506 of FIG. 5). The ssDNA was contacted with a primary antibody or dye that binds ssDNA for binding (phase 508 of FIG. 5). The ssDNA/primary antibody (or dye) complex was then contacted with a HRP-linked secondary antibody for binding (phase 510 of FIG. 5). The ssDNA/primary antibody (or dye)/HRP-linked secondary antibody complex was then contacted with a substrate such that HRP initiates polymerization of the substrate resulting in formation of non-soluble precipitates on the surface of the BLI biosensor. The precipitates introduce thickness changes on the surface of the BLI biosensor that correspond to signal (nm) shift for (phase 512 of FIG. 5). BLI was used to measure the signal shift (nm) over time to detect signals generated by the HRP and the substrate, with a positive shift indicating ssDNA binding to the biosensor.

The fourth method is depicted in FIGS. 6 and 7. In the fourth method, the BLI biosensor loaded with negatively charged biopolymer was equilibrated (phase 702 of FIG. 7), and then was loaded with polyethylenimine (PEI) (phase 704 of FIG. 7), with appropriate washes. Heat stress was applied to samples each containing AAV8 with the full capsid ratio of 0%, 30%, 46%, 62%, and 90%, respectively, to allow ssDNA contained in the viral capsids to be released and bound to the biosensor (phase 706 of FIG. 7). The ssDNA was contacted with a HRP-capture antibody that binds ssDNA to allow for binding (phase 708 of FIG. 7). The ssDNA/HRP-linked antibody complex was then contacted with a substrate such that HRP catalyzes the conversion of the substrate for the detection of ssDNA (phase 710 of FIG. 7). BLI was used to measure the signal shift (nm) over time to detect signals generated by the HRP and the substrate, with a positive shift indicating ssDNA binding to the biosensor.

In all of the four detection methods described herein, samples with different AAV full/empty capsid ratios showed different binding kinetics, indicating that any of the four methods can be used to quantitate the full/empty capsid ratio in a sample.

### Example 2: Binding Kinetics of Intact Adeno-Associated Virus (AAV) Capsids with Varied Titer but Same Empty/Full Capsid Ratio to a Bio-Layer Interferometry (BLI) Biosensor and Associated Interferometry Signals

This example relates to binding kinetics of AAV8 capsids to the bio-layer interferometry (BLI) biosensor in samples with varied AAV8 capsid titers, but the same empty/full capsid ratios.

The BLI biosensor preloaded with negatively charged biopolymer, was then loaded with polyethylenimine (PEI). Samples each containing full AAV8 capsids at the concentration (titer) of 5×10¹¹ vg/ml, 2.5×10¹¹ vg/ml, 1.25×10¹¹ vg/ml, 6.25×10¹¹ and 3.125×10¹⁰ vg/ml, respectively, were measured over time using BLI for a signal shift (nm), with a positive shift indicating binding of viral capsids to the biosensor. As shown in FIG. 8, the binding of AAV8 capsids to the biosensor showed positive correlation between binding signal and capsid titer within loading time frame. This can be explained by more AAV8 particles in samples with a higher titer resulting in faster and higher binding towards positively charged biosensor.

### Example 3: Binding Kinetics of Intact Adeno-Associated Virus (AAV) Capsids with Same Titer but Varied Empty/Full Ratio to a Bio-Layer Interferometry (BLI) Biosensor and Associated Interferometry Signals

This example relates to binding kinetics of intact AAV2 capsids to the BLI biosensor in samples with same viral titer but varied empty/full capsid ratio.

The BLI biosensor preloaded with negatively charged biopolymer, was then loaded with polyethylenimine (PEI). Signal shift (nm) was measured over time using BLI for samples each containing 2×10¹¹ vg/ml AAV2 with the empty capsid ratio of 10%, 17%, 26%, 36%, 50%, 63%, 70%, and 82.7%, respectively, with a shift indicating binding of viral capsids to the biosensor. As shown in FIG. 9, in samples with a lower empty capsid ratio, the binding of AAV2 capsids (empty and full) to the biosensor reached saturation faster as compared to samples with a higher empty capsid ratio. This can be explained by the higher negative charges of a full capsid with a DNA core as compared to an empty capsid without a DNA core.

1. A method for quantitating one or more polynucleotides contained within one or more viral capsids in a sample, the method comprising:
subjecting the sample containing one or more viral capsids to a separation process to separate the one or more polynucleotides from the one or more viral capsids, the sample being a subject sample or a reference sample;
contacting the sample with a biosensor such that the one or more polynucleotides in the sample are bound to a positively charged biopolymer of the biosensor, the biosensor containing a core component, a negatively charged biopolymer bound to an external surface of the core component, and the positively charged biopolymer bound to an external surface of the negatively charged biopolymer;
contacting the one or more polynucleotides bound to the biosensor with one or more signal development elements to generate signals indicative of an amount of the one or more polynucleotides bound to the biosensor; and
determining a ratio of empty and full viral capsids in the subject sample based at least partially on the amount of polynucleotides in the reference sample bound to the biosensor and an amount of polynucleotides in the subject sample bound to the biosensor.

2. The method of claim 1, wherein the separation process comprises applying heat to the one or more viral capsids, thereby separating the one or more polynucleotides from the one or more viral capsids.

3. The method of claim 1, wherein the one or more polynucleotides comprise single stranded DNA (ssDNA).

4. The method of claim 1, wherein the one or more viral capsids comprise capsids of adeno-associated virus (AAV).

5. The method of claim 1, wherein:
the core component comprises epoxypropylsilane or aminopropylsilane;
the negatively charged biopolymer comprises one or more of DNA, dextran, and carboxylic acid (COOH)-functionalized molecule; and/or
the positively charged biopolymer comprises one or more of polyethylenimine (PEI), chitosan, poly-L-lysine, and polyallylamine.

6. The method claim 1, wherein the one or more signal development elements comprise a dye, an antibody, or an antibody linked to a first enzyme.

7. The method of claim 6, wherein the dye is a cyanine dye, ethidium bromide, propidium iodide, crystal violet, a dUTP-conjugated probe, DAPI (4′,6-diamidino-2-phenylindole), 7-aminoactinomycin D (7-AAD), a Hoechst dye, a CYBR® dye, or EVAGREEN®.

8. The method of claim 6, wherein the antibody is a nucleic acid antibody, a ssDNA antibody, or a ss/dsDNA specific antibody, and the enzyme is horseradish peroxidase (HRP), alkaline phosphatase (AP), β-galactosidase, acetylcholinesterase, or catalase.

9. The method of claim 6, wherein the signals are generated by contacting the one or more signal development elements comprising the antibody linked to the first enzyme with a substrate; or by contacting the one or more signal development elements comprising the antibody with a secondary antibody linked to a second enzyme, and contacting the second enzyme with a substrate.

10. (canceled)

11. A method for quantitating one or more polynucleotides contained within one or more viral capsids in a sample, the method comprising:
subjecting the sample containing one or more viral capsids to a separation process to separate the one or more polynucleotides from the one or more viral capsids;
contacting the sample with a biosensor such that the one or more polynucleotides in the sample are bound to a positively charged biopolymer of the biosensor, the biosensor containing a core component, a negatively charged biopolymer bound to an external surface of the core component, and the positively charged biopolymer bound to an external surface of the negatively charged biopolymer, different full/empty viral capsid ratios in the sample resulting in different binding kinetics of the biosensor to the one or more polynucleotides contained in the sample; and
contacting the one or more polynucleotides bound to the biosensor with one or more signal development elements to generate signals indicative of an amount of the one or more polynucleotides bound to the biosensor.

12. The method of claim 11, comprising determining a ratio of empty and full viral capsids based at least partially on the binding kinetics of the biosensor to the one or more polynucleotides.

13. The method of claim 1, wherein the signals are indicative of spectral shift measured using bio-layer interferometry (BLI).

14. The method of claim 13, wherein a positive spectral shift indicates binding of the one or more polynucleotides to the biosensor.

15. A method for quantitating one or more viral capsids of adeno-associated virus (AAV) in a sample, the method comprising:
contacting the sample with a biosensor such that the one or more viral capsids in the sample are bound to a positively charged biopolymer of the biosensor and generate a first set of signals indicative of binding of the viral capsids to the biosensor, the biosensor containing a core component, a negatively charged biopolymer bound to an external surface of the core component, and the positively charged biopolymer bound to an external surface of the negatively charged biopolymer; and
quantitating the first set of signals generated by binding of the viral capsids to the biosensor.

16. The method of claim 15, wherein the first set of signals are indicative of an amount of the viral capsids bound to the biosensor and/or a full/empty viral capsid ratio based at least partially on binding kinetics of the biosensor to the viral capsids.

17. The method of claim 15, wherein the first set of signals are indicative of spectral shift measured using bio-layer interferometry (BLI).

18. The method of claim 17, wherein a positive spectral shift indicates binding of the one or more viral capsids to the biosensor.

19. The method of claim 15, further comprising:
subjecting the one or more viral capsids bound to the biosensor to a separation process to separate the polynucleotides from the one or more viral capsids;
contacting the polynucleotides with the biosensor to bind the polynucleotides to the positively charged biopolymer of the biosensor; and
contacting the polynucleotides bound to the biosensor with one or more signal development elements to generate a second set of signals indicative of an amount of the polynucleotides bound to the biosensor.

20. A method for quantitating polynucleotides contained within one or more viral capsids of adeno-associated virus (AAV) in a sample, the method comprising:
contacting the one or more viral capsids with a first biosensor to isolate and/or purify the viral capsids from a crude sample, the first biosensor comprising an affinity biosensor;
subjecting the one or more viral capsids bound to the first biosensor to a separation process to separate the polynucleotides from the one or more viral capsids;
contacting the polynucleotides with a second biosensor such that the polynucleotides are bound to the second biosensor;
contacting the polynucleotides bound to the second biosensor with one or more signal development elements to generate signals indicative of an amount of the polynucleotides bound to the biosensor, wherein the one or more signal development elements comprise a dye; and
determining a ratio of empty and full viral capsids based at least partially on the amount of polynucleotides bound to the second biosensor and/or binding kinetics of the second biosensor to the polynucleotides.

21. The method of claim 20, wherein the first biosensor is an AAV antibody-based affinity biosensor.

22. The method of claim 20, wherein the second biosensor comprises a core component, a negatively charged biopolymer bound to an external surface of the core component, and the positively charged biopolymer bound to an external surface of the negatively charged biopolymer.
